SEQUENCE LISTING (28 sequences)

SEQ ID NO: 1 - protein, 243 aa, Triticum aestivum
MAASEQSSESSSTSCTSSYSTSSCSPRADDKKRNGGGKRKRAAAADEEPAPAAAAASYRGVRMRAWGKWVSEIREPRKKSRIWLGTFPCPEMAARAHDAAALSIKGARAVLNFPDLAPALPRPASLAPCDVQAAAARAALMHDHDHQCCSASTSPAAAPDSARGNAPAPCDQPAARDEPEHEPPFQPSQGNEQQLTAAQVEMVFDELAPLWVENVVDFAPSDHCWTAYDCLDPIGFQPLLWEY

SEQ ID NO: 2 - protein, 237 aa, Brachypodium distachyon
MAASEQSSDSNSSTTTTTSSSCSASSCSPRSTPQTQHGDNKRKRARVEASASASANYRGVRMRAWGKWVSEIREPRKKSRIWLGTFPCPEMAARAHDAAALCVKGARAVLNFPDLAHALPRPASLAPRDVQAAAARAALMHPERAEEGAIDDDDAPLAGGGEPSPSPSPETKENEPSRGSEETAEAEAEAQELVFDELAPLWVEDVVDFAPSDHCWTAAYDALGLDPVGFQPLLWEY

SEQ ID NO: 3 - protein, 228 aa, Oryza sativa
MAASEQSSESSSTASTSSCGKKQQVAGKRKREDVGGGGEQAAAVAYRGVRMRAWGKWVSEIREPRKKSRIWLGTFPCPEMAARAHDVAALSIKGARAVLNFPDLAPALPRPASLAPRDVQAAAALAAVMHHHKHPSSSTSTSSPPAAPPPDEHHPRHEPQQPESSREDDQQQQPAAAAAAQMAVAELVFDELAPLWVEDVVEFGTSDHCWTAYDALDPIGFQPLLWEY

SEQ ID NO: 4 - protein, 251 aa, Sorghum bicolor
MAMSELSSDTSSTSSYSSDPSTSTSPRAAAPCSAAAAVSGKKRPRSNGGGESGSAPAYRGVRMRAWGKWVSEIREPRKKSRIWLGTFPCPEMAARAHDVAALSIKGARAVLNFPDLARHLPRPASLAPRDVQAAAARAALMHMHSDGAGSGSGSGGSSSPSSSSSSSTRDAADTDGAAAHDEPEPEPEPQRQHERQPVEMAAELVFDELAPLWVEDVVEFGGPPSDHTWTPCDGLDAAVGFLHPLPLLWDY

SEQ ID NO: 5 - protein, 255 aa, Zea mays
MAMSEPSSDTSCTSSCSSGPSTSTSPRAAPSAAAAAASGGGKKRPRSDDGGSSSSSAPAYRGVRMRAWGKWVSEIREPRKKSRIWLGTFPCPEMAARAHDAAALSVKGARAVLNFPGLARHLPRPASLAPRDVQAAAARAALMHHSSAGCCCCGGGGGGGSTATATSSSSSSSAAADVGGCGQRRDGAAAHDEPEPERQPLEVAAELVFDELAPLWVEDVVELGPSADHPWPWTPCDGLDIAAGFHPLPLLWDDY

SEQ ID NO: 6 - protein, 238 aa, Setaria italica
MSDLSSDTSSTSSYSPSPSTSSSSRGGGGGGKKRPREPAGDVPAYRGVRMRAWGKWVSEIREPRKKSRIWLGTFPCPEMAARAHDVAALSIKGARAVLNFPDLAPALPRPTSLAPRDVQAAAARAALMHHKHQKSGGGGSSSSSSSSSAAPAAGSAAAAADDAQRRTPGPAAHRHDDPEPEPSREERQPAQVEVAELVFDELAPLWVEDVVEFGPSDHPWTPYDGLDAVGFQPLLWEY

SEQ ID NO: 7 - protein, 237 aa, Zea mays
MAASEQSSDSNSSTTTTTSSSCSASSCSPRSTPQTQHGDNKRKRARVEASASASANYRGVRMRAWGKWVSEIREPRKKSRIWLGTFPCPEMAARAHDAAALSIKGARAVLNFPDLAPALPRPASLAPCDVQAAAARAALMHHHKHPSSSTSTSSPPLAGGGEPSPSPSPETKENEPSRGSEETAEAEAEAQELVFDELAPLWVEDVVDFAPSDHCWTAAYDALGLDPIGFQPLLWEY

SEQ ID NO: 8 - protein, 246 aa, Zea mays
MAASEQSSDSNSTTTSSSCSASSCSPRSTPKKRNGDNKRKRAAAAARVEASASASANYRGVRMRAWGKWVSEIREPRKKSRIWLGTFPCPEMAARAHDAAALSIKGARAVLNFPDLAPALPRPASLAPCDVQAAAARAALMHDHDHQCCSTSTSPAASPPLAGGGEPSPSPSPETKEDEPEPEPEPSRGSQRQHERQPVEMAAELVFDELAPLWVENVVDFAPSDHCWTAAYGLDPIGFQPLLWEY

SEQ ID NO: 9 - protein, 245 aa, Zea mays
MAASEQSSDSNSTTTCSSYSASSCSPRSDDKKRNGGGKRKRAAAADEEPAPASASANYRGVRMRAWGKWVSEIREPRKKSRIWLGTFPCPEMAARAHDAAALSIKGARAVLNFPDLAPALPRPASLAPCDVQAAAARAALMHDHDHQCCSTSTSPAASPDSARGGAPCDQPSPAARDEPEPEPEPSRGSQRQHERQPVEMAAELVFDELAPLWVENVVDFAPSDHCWTAYDCLDPIGFQPLLWEY

SEQ ID NO: 10 - protein, 243 aa, Zea mays
MAASEQSSDSNSTTTCSSYSASSCSPRSDDKKRNGGGKRKRAAAADEEPAPASASANYRGVRMRAWGKWVSEIREPRKKSRIWLGTFPCPEMAARAHDAAALSIKGARAVLNFPDLAPALPRPASLAPCDVQAAAARAALMHDHDHQCCSTSTSPAASPDSARGNAPCDQPSPAARDEPEPEPPFQPSQGNEQQLTAAQVELVFDELAPLWVENVVDFAPSDHCWTAYDCLDPIGFQPLLWEY

SEQ ID NO: 11 - protein, 243 aa, Zea mays
MAASEQSSDSNSTTTCSSYSASSCSPRSDDKKRNGGGKRKRASAADEEPAPASASANYRGVRMRAWGKWVSEIREPKKKSRIWLGTFPTPEMAARAHDAAALSVKGARAVLNFPDLAHALPRPASLAPRDVQAAAARAAVMHDHDHQCCSTSTSPAASPDSARGNAPCDQPSPAARDEPEPEPPFQPSQGNERQLTAAQVELVFDELAPLWVEDVVDFAPSDHCWTAYDCLDPVGFQPLLWEY

SEQ ID NO: 12 - DNA, 732 bp, Triticum aestivum
atggcggcgagcgagcagagctccgagtcctcgtcgacgtcctgcacttcctcctactccacgtcctcgtgctcccccagggccgacgacaagaagcggaacggcggcggcaagcggaagcgcgcagcggcggccgacgaggagccggccccggcggcggcggcggcgtcgtatcgcggcgtgcggatgcgggcgtggggcaagtgggtgtcggagatcagggagccgcgcaagaagtcccgcatctggctcggcaccttcccgtgcccggagatggccgcgcgcgcgcacgacgccgccgcgctcagcatcaagggcgcccgcgccgtgcttaacttcccggacctcgcgcccgcgctccctcgcccggcctccctcgccccgtgcgacgtccaggccgccgccgcgcgcgccgccctcatgcacgaccacgaccaccagtgctgctccgcctcaacctcccccgccgccgcgccggactccgcgcgcggcaatgccccggcgccatgcgaccagccggccgcgcgagacgagccggagcatgagccgccgttccagccgagccagggaaatgagcagcagctcacggctgcacaggtggagatggtgttcgacgagctggcgccgctgtgggtggagaacgtggtggatttcgcgccgtccgatcactgctggacggcgtacgattgtctcgacccaatcggcttccagcctctcctgtgggagtattag

SEQ ID NO: 13 - DNA, 1680 bp, Oryza sativa
atccctcagccgcctttcactatcttttttgcccgagtcattgtcatgtgaaccttggcatgtataatcggtgaattgcgtcgattttcctcttataggtgggccaatgaatccgtgtgatcgcgtctgattggctagagatatgtttcttccttgttggatgtattttcatacataatcatatgcatacaaatatttcattacactttatagaaatggtcagtaataaaccctatcactatgtctggtgtttcattttatttgcttttaaacgaaaattgacttcctgattcaatatttaaggatcgtcaacggtgtgcagttactaaattctggtttgtaggaactatagtaaactattcaagtcttcacttattgtgcactcacctctcgccacatcaccacagatgttattcacgtcttaaatttgaactacacatcatattgacacaatattttttttaaataagcgattaaaacctagcctctatgtcaacaatggtgtacataaccagcgaagtttagggagtaaaaaacatcgccttacacaaagttcgctttaaaaaataaagagtaaattttactttggaccacccttcaaccaatgtttcactttagaacgagtaattttattattgtcactttggaccaccctcaaatcttttttccatctacatccaatttatcatgtcaaagaaatggtctacatacagctaaggagatttatcgacgaatagtagctagcatactcgaggtcattcatatgcttgagaagagagtcgggatagtccaaaataaaacaaaggtaagattacctggtcaaaagtgaaaacatcagttaaaaggtggtataaagtaaaatatcggtaataaaaggtggcccaaagtgaaatttactcttttctactattataaaaattgaggatgtttttgtcggtactttgatacgtcatttttgtatgaattggtttttaagtttattcgcttttggaaatgcatatctgtatttgagtcgggttttaagttcgtttgcttttgtaaatacagagggatttgtataagaaatatctttaaaaaaacccatatgctaatttgacataatttttgagaaaaatatatattcaggcgaattctcacaatgaacaataataagattaaaatagctttcccccgttgcagcgcatgggtattttttctagtaaaaataaaagataaacttagactcaaaacatttacaaaaacaacccctaaagttcctaaagcccaaagtgctatccacgatccatagcaagcccagcccaacccaacccaacccaacccaccccagtccagccaactggacaatagtctccacacccccccactatcaccgtgagttgtccgcacgcaccgcacgtctcgcagccaaaaaaaaaaaaagaaagaaaaaaaagaaaaagaaaaaacagcaggtgggtccgggtcgtgggggccggaaacgcgaggaggatcgcgagccagcgacgaggccggccctccctccgcttccaaagaaacgccccccatcgccactatatacatacccccccctctcctcccatccccccaaccctaccaccaccaccaccaccacctccacctcctcccccctcgctgccggacgacgagctcctcccccctccccctccgccgccgccgcgc

SEQ ID NO: 14 - DNA, 860 bp, Zea mays
ctggtaattattggctgtaggattataaacagagcctaaatagctggaatagctctagccctcaatccaaactaatgatatctatacttatgcaactctaaatttttattctaaaagtaatatttcatttttgtcaacgagattctctactctattccacaatcttttgaagcaatatttaccttaaatctgtactctataccaataatcatatattctattatttatttttatctctctcctaaggagcatccccctatgtctgcatggcccccgcctcgggtcccaatctcttgctctgctagtagcacagaagaaaacactagaaatgacttgcttgacttagagtatcagataaacatcatgtttacttaactttaatttgtatcggtttctactatttttataatatttttgtctctatagatactacgtgcaacagtataatcaacctagtttaatccagagcgaaggattttttactaagtacgtgactccatatgcacagcgttccttttatggttcctcactgggcacagcataaacgaaccctgtccaatgttttcagcgcgaacaaacagaaattccatcagcgaacaaacaacatacatgcgagatgaaaataaataataaaaaaagctccgtctcgataggccggcacgaatcgagagcctccatagccagttttttccatcggaacggcggttcgcgcacctaattatatgcaccacacgcctataaagccaaccaacccgtcggaggggcgcaagccagacagaagacagcccgtcagcccctctcgtttttcatccgccttcgcctccaaccgcgtgcgctccacgcctcctccaggaaagcga

SEQ ID NO: 15 - protein, 242 aa, Nassella lepida
MAASEQSSESSSTSYTSSYSTSSCSPRADDKKQSGGSKRKRAAADEEPAPAAAAAASYRGVRMRAWGKWVSEIREPRKKSRIWLGTFPCPEMAARAHDAAALSIKGARAVLNFPDLAPALPRPASLAPCDVQAAAARAALMHDHDHQCCSASTSPAAAPASPRGNAPAHCDQPAARDEPAHEPFQPSQGNEQQLTAAQVEMVFDELAPLWVEDVVDFAPSDHCWTTYDCLDPIGFQPLLWEY

SEQ ID NO: 16 - DNA, 729 bp, Nassella lepida
atggcggcgagcgagcagagctccgagtcctcgtcgacgtcctacacttcctcctactccacgtcctcgtgctcgcccagggccgacgacaagaagcagagcggcggcagtaagcggaagcgcgcggcggccgacgaggagccggctccggcggcggcggcggcggcgtcgtaccgcggcgtgcggatgcgggcgtggggcaagtgggtgtcggagatcagggagccgcgcaagaagtcccgcatctggctcggcaccttcccgtgcccggagatggccgcgcgcgcccacgacgccgccgcgctcagcatcaagggcgcccgcgccgtgctcaacttcccggacctcgcgcccgcgctcccccgcccggcctccctcgccccgtgcgacgtccaggccgccgccgcgcgcgccgccctcatgcacgaccacgaccaccagtgctgctctgcttcaacctcccccgccgccgctcccgcctccccgcgcggcaatgctccggcgcattgcgaccagccggccgcgcgagacgaaccggcgcatgagccgttccagccgagccagggaaatgagcagcagctgacggctgcacaggtggagatggtgttcgacgagctggcgccgctgtgggtggaggacgtggtggatttcgcgccgtccgatcactgctggacgacgtacgattgtctcgacccaatcggcttccagcctctcctgtgggagtattag

SEQ ID NO: 17 - protein, 242 aa, Bromus ciliatus
MAASEQSSESSSTSYTSSYSTSSCSPRADDKKQNAGNKRKRAAADEEPAPAPAAAASYRGVRMRAWGKWVSEIREPRKKSRIWLGTFPCPEMAARAHDAAALSIKGARAVLNFPDLAPALPRPASLAPCDVQAAAARAALMHDHDNQCCSASTSPAAAPASARGNAPAPCDQPAARDEPAHEPFQPSQGNEQQLTAVQVEMVFDELAPLWVEDVVDFAPSDHCWTAYDCLDPIGFQPLLWEY

SEQ ID NO: 18 - DNA, 729 bp, Bromus ciliatus
atggcggcgagcgagcagagctcggagtcctcgtcgacgtcctacacttcctcctactccacgtcctcgtgctcgcccagggccgacgacaagaagcagaacgccggcaacaagcggaagcgcgcggcggccgacgaggagccggcccctgcgcctgcggcggcggcgtcgtaccgcggcgtgcggatgcgggcgtggggcaagtgggtgtcggagatcagggagccgcgcaagaagtcccgcatctggctcggcaccttcccgtgcccggagatggccgcgcgcgcccacgacgccgccgcgctcagcatcaagggcgcccgcgcggtgctcaacttcccggacctcgcgccagcgctcccccgcccggcctccctcgccccgtgcgacgtccaggccgctgccgcgcgcgccgccctcatgcacgaccacgacaatcagtgctgctctgcttcaacctcccccgccgccgctcccgcctccgcgcgcggcaatgctccggcgccatgcgaccagccggccgcgcgagacgagccggcgcatgagccgttccagccgagccagggaaatgagcagcagctgacggccgtacaggtggagatggtgttcgacgagctggcgccgctgtgggtggaggacgtggtggatttcgcgccgtccgatcactgctggacggcgtacgattgtctcgacccaataggcttccagcctctcctgtgggagtattag

SEQ ID NO: 19 - protein, 248 aa, Deschampsia cespitosa
MAASEQSSESSSTTATTTSSYTSSYSTSSCSPRADDKKQDGGNKRKRAAADEEAAPAAAASYRGVRMWAWGKWVSEIREPRKKSRIWLGTFPCPEMAARAHDAAALSIKGARAVLNFPDLAPALPRPASLAPCDVQAAAARAALMHDHDHQCCSASTSPAAAPASPCGNAPAPSDQPATRDEPEPEHEPFQPSQGNEQQLTATQVEMVFDELAPLWVEDVVDLTPSDHCWTAYDCLDPIGFQPLLWEY

SEQ ID NO: 20 - DNA, 747 bp, Deschampsia cespitosa
atggcggcgagcgagcagagctccgagtcctcgtcgacgacggcgacgaccacgtcgtcctacacttcctcctactccacgtcctcgtgctcgcccagggccgacgacaagaagcaggacggcggcaacaagcggaagcgcgcggcggccgacgaggaggcggccccggcggcggcggcgtcgtaccgcggcgtgcggatgtgggcgtggggcaagtgggtgtcggagatcagggagccgcgcaagaagtcccgcatctggctcggcaccttcccgtgcccggagatggccgcgcgcgcccacgacgccgccgcgctcagtatcaagggcgcacgcgcggtgctcaacttcccggacctcgcgcccgcgctcccccgtccggcctccctcgccccgtgcgacgtccaggccgccgccgcgcgcgccgccctcatgcacgaccacgaccaccagtgctgctccgcttcaacctcccccgccgccgcgccggcctccccgtgcggcaatgccccggcgccaagcgaccagccggccacgcgagacgagccggagccggagcatgagccgttccagccgagccagggaaatgagcagcagctcacggctacgcaggtggagatggtgttcgacgagctggcgccgctgtgggtggaggacgtggtggatttaacgccgtccgatcactgctggacggcgtacgattgtctcgacccaatcggcttccagcctctcctgtgggagtattag

SEQ ID NO: 21 - protein, 251 aa, Bromus kalmii
MAASEQSSESSSTTTNTTTSSYTSSYSTSSCSPRPDDKKQSGGNKRKRAEADEEAAPPAAAAAASYRGVRMRAWGKWVSEIREPRKKSRIWLGTFPCPEMAARAHDAAALSIKGARAVLNFPDLAPALPRPASLAPCDVQAAAARAALMHDHDNQCCSASTSPAAAPAYTRGNAPAPCDEPAALDEPEHESPFQPSQGNDLQLTAAQVEMVFDELAPLWVEDVVDFAPSDHCWTAYDCLDPIGFQPLLWEY

SEQ ID NO: 22 - DNA, 756 bp, Bromus kalmii
atggcggcgagcgagcagagctccgagtcctcgtcgacgacgaccaacacgaccacgtcgtcctacacctcctcctactccacgtcctcgtgctcccccaggcccgacgacaagaagcagagcggcggcaacaagcggaagcgcgcggaggccgacgaggaggcggccccaccggcggcggcggcggcggcgtcgtaccgcggcgtgcggatgcgggcgtggggcaagtgggtgtcggagatcagggagccgcgcaagaagtcccgcatctggctcggcaccttcccgtgcccggagatggccgcgcgcgcgcacgacgccgccgcgctcagcatcaagggcgcccgcgccgtgctcaacttcccggacctcgcgcccgcgctcccccgcccggcctccctcgccccgtgcgacgtccaggccgccgccgcgcgcgccgccctcatgcacgaccacgacaaccagtgctgctccgcctcaacctcccccgccgccgctcccgcctacacgcgcggcaatgccccggcgccatgcgacgagccggccgcgctagacgagcccgagcatgagtcgccgttccagccgagccagggaaatgacctgcagctcacggctgcacaggtggagatggtgttcgacgagctggcgccgctgtgggtggaggacgtggtggatttcgcgccgtccgatcactgctggacggcgtacgattgtctcgacccaatcggcttccagcctctcctgtgggagtattag

SEQ ID NO: 23 - protein, 248 aa, Nassella lepida
MAASEQSSESSSTTTNTTTSSYTSSYSTSSCSPRADDKKQSGGNKRKRAEADEVAAPPAAASYRGVRMRAWGKWVSEIREPRKKSRIWLGTFPCPEMAARAHDAAALSIKGARAVLNFPDLAPALPRPASLAPCDVQAAAARAALMHDHDNQCCSASTSPAAAPASARGNAPAPCDEPAALDEPEHEPPFQPSQGNDLQLTAAQVEMVFDELAPLWVEDVVDFAPSDHCWTAYDCLDPIGFQPLLWEY

SEQ ID NO: 24 - DNA, 747 bp, Nassella lepida
atggcggcgagcgagcagagctcggagtcctcctcgacgacgaccaacacgaccacgtcgtcctacacttcctcctactccacgtcctcgtgctcccccagggccgacgacaagaagcagagcggcggcaacaagcggaagcgcgcggaggccgacgaggtggcggccccaccggcggcggcgtcgtaccgcggcgtgcggatgcgggcgtggggcaagtgggtgtcggagatcagggagccgcgcaagaagtcccgcatctggctcggcaccttcccgtgcccggagatggccgcgcgcgcgcacgacgccgccgcgctcagcatcaagggcgcccgcgccgtgctcaacttcccggacctcgcgcccgcgctcccccgcccggcctccctcgccccgtgcgacgtccaggccgccgccgcgcgcgccgccctcatgcacgaccacgacaaccagtgctgctccgcctcaacctcccccgccgccgctcccgcctccgcgcgcggcaatgccccggcgccatgcgacgagccggccgcgctagacgagcccgagcatgagccgccgttccagccgagccagggaaatgacctgcagctcacggctgcacaggtggagatggtgttcgacgagctggcgccgctgtgggtggaggacgtggtggatttcgcgccgtccgatcactgctggacggcgtacgattgtctcgacccaatcggcttccagcctctcctgtgggagtattag

SEQ ID NO: 25 - protein, 245 aa, Elymus californicus
MAASEQSSESSSTTTTTTTSSYTSSYSTSSCSPRAGDKKQNGNKRKRAADEEAAPAAAASYRGVRMRAWGKWVSEIREPRKKSRIWLGTFPCPEMAARAHDAAALSIKGARAVLNFPDLAPALPRPASLAPCDVQAAAARAALMHDHDHQCCSARTSPAAALASARDNAPASCDLPAARDEPEQEPFQPSQGNEQQLTTAQVEMVFDELAPLWVEDVVDFAPSDHCWTAYDCLDPIGFQPLLWEY

SEQ ID NO: 26 - DNA, 738 bp, Elymus californicus
atggcggcgagcgagcagagctcggagtcctcgtcgacgacgacgacgacgaccacgtcctcctacacatcctcctactccacgtcctcgtgctcgcccagggccggcgacaagaagcagaacggcaacaagcggaagcgcgcggccgacgaggaggcggccccggcggcggcggcgtcgtaccgcggcgtgcggatgcgggcgtggggcaagtgggtgtcggagatcagggagccgcgcaagaagtcccgcatctggctcggcaccttcccgtgcccggagatggccgcgcgcgcccacgacgccgccgcgctcagcatcaagggcgcccgcgcggtgctcaacttcccggacctcgcgcccgcgctcccgcgcccggcctccctcgccccgtgcgacgtccaggccgccgccgcgcgcgccgccctcatgcacgaccacgaccaccagtgctgctctgctcgaacctcccccgccgccgcgctggcctccgcgcgggacaatgccccggcgtcatgcgacctgccggccgcgcgagacgagccggagcaggagccgttccagccgagccagggaaatgagcagcagcttacgactgcacaggtggagatggtgttcgacgagctggcgccgctgtgggtggaggacgtggtggatttcgcaccgtccgatcactgctggacggcgtacgattgtcttgacccaatcggcttccagcctctcctgtgggagtattag

SEQ ID NO: 27 - protein, 248 aa, Achnatherum lettermanii
MAASEQSSESSSTTATTTSSYTSSYSTSPCSPRADDKKQDGGNKRKRAAADEEAAPAAAASYRGVRMRVWGKWVSEIREPRKKPRIWLGTFPCPEMAARAHDAAALSIKGARAVLNFPDLAPALPRPASLAPCDVQAAAARAALMHDHDHQCCSASTSPAAAPASPCGNAPAPSDQPATRDGPEPEHEPFQPSQGNEQQLTATQVEMVFDELAPLWVEDVVDLTPSDHCWTAYDRLDPIGFQPLLWEY

SEQ ID NO: 28 - DNA, 747 bp, Achnatherum lettermanii
atggcggcgagcgagcagagctccgagtcctcgtcgacgacggcgacgaccacgtcgtcctacacttcctcctactccacgtccccgtgctcgcccagggccgacgacaagaagcaggacggcggcaacaagcggaagcgcgcggcggccgacgaggaggcggccccggcggcggcggcgtcgtaccgcggcgtgcggatgcgggtgtggggcaagtgggtgtcggagatcagggagccgcgcaagaagccccgcatctggctcggcaccttcccgtgcccggagatggccgcgcgcgcccacgacgccgccgcgctcagtatcaagggcgcacgcgcggtgctcaacttcccggacctcgcgcccgcgctcccccgtccggcctccctcgccccgtgcgacgtccaggccgccgccgcgcgcgccgccctcatgcacgaccacgaccaccagtgctgctccgcttcaacctcccccgccgccgcgccggcctccccgtgcggcaatgccccggcgccaagcgaccagccggccacgcgagacgggccggagccggagcatgagccgttccagccgagccagggaaatgagcagcagctcacggctacgcaggtggagatggtgttcgacgagctggcgccgctgtgggtggaggacgtggtggatttaacgccgtccgatcactgctggacggcgtacgatcgtctcgacccaatcggcttccagcctctcctgtgggagtattag

(19) United States
(12) Patent Application Publication
(10) Pub. No.: US 2018/0105824 A1
(43) Pub. Date: Apr. 19, 2018

(54) MODULATION OF DREB GENE EXPRESSION TO INCREASE MAIZE YIELD AND OTHER RELATED TRAITS

(71) Applicant: PIONEER HI-BRED INTERNATIONAL, INC., JOHNSTON, IA (US)

(72) Inventors: CARL SIMMONS, DES MOINES, IA (US); SOBHANA SIVASANKAR, ADEL, IA (US)

(73) Assignee: PIONEER HI-BRED INTERNATIONAL, INC., JOHNSTON, IA (US)

(21) Appl. No.: 15/561,388

(22) PCT Filed: Mar. 22, 2016
(86) PCT No.: PCT/US16/23530

§ 371 Date: Sep. 25, 2017

Related U.S. Application Data
(60) Provisional application No. 62/138,540, filed on Mar. 26, 2015.

(51) Int. Cl. C12N 15/82; C07K 14/415

(52) U.S. Cl. CPC C12N 15/8217; C12N 15/8279; C07K 14/415; C12N 15/8273

(57) ABSTRACT

Nucleotide sequences encoding DREB2 polypeptides are provided herein, along with plants and cells having increased levels of DREB2 gene expression, increased levels of DREB2 transcription factor activity, or both. Plants with increased levels of at least one DREB2 gene expression that exhibit increased yield, increased abiotic stress tolerance, or any combination of these, are provided. Methods for increasing yield, and abiotic stress tolerance in plants, by modulating DREB2 gene expression or activity, are also provided.

### CROSS-REFERENCE TO RELATED APPLICATIONS

This application claims priority to U.S. Provisional Application Ser. No. 62/138,540, filed Mar. 26, 2015, incorporated herein by reference in its entirety.

### REFERENCE TO SEQUENCE LISTING SUBMITTED ELECTRONICALLY

The official copy of the sequence listing is submitted electronically via EFS-Web as an ASCII formatted sequence listing with a file named 6763_PCT_Filing_ST25 created on Mar. 10, 2016 and having a size of 28 kilobytes and is filed concurrently with the specification. The sequence listing contained in this ASCII formatted document is part of the specification and is herein incorporated by reference in its entirety.

### FIELD

The field relates to plant breeding and genetics and, in particular, to recombinant DNA constructs useful in plants for increasing yield and/or conferring tolerance to abiotic stress tolerance.

### BACKGROUND

Yield is a trait of particular economic interest, especially because of increasing world population and the dwindling supply of arable land available for agriculture. Crops such as corn, wheat, rice, canola and soybean account for over half the total human caloric intake, whether through direct consumption of the seeds themselves or through consumption of meat products raised on processed seeds.

Abiotic stress is the primary cause of crop loss worldwide, causing average yield losses of more than 50% for major crops (Boyer, J. S. (1982) Science 218:443-448; Bray, E. A. et al. (2000) In Biochemistry and Molecular Biology of Plants, Edited by Buchannan, B. B. et al., Amer. Soc. Plant Biol., pp. 1158-1203).

Among the various abiotic stresses, drought is a major factor that limits crop productivity worldwide. Another abiotic stress that can limit crop yields is low nitrogen stress. The adsorption of nitrogen by plants plays an important role in their growth. Plants synthesize amino acids from inorganic nitrogen in the environment. Consequently, nitrogen fertilization has been a powerful tool for increasing the yield of cultivated plants, such as maize and soybean. If the nitrogen assimilation capacity of a plant can be increased, then increases in plant growth and yield increase are also expected. Plant varieties that have tolerance to drought stress and/or better nitrogen use efficiency (NUE) are desirable. In addition, yield increase under normal crop growing conditions are also desirable.

### SUMMARY

The present disclosure includes:

One embodiment is a maize plant in which expression of a DREB2 gene is increased, when compared to a control plant, wherein the DREB2 gene encodes a DREB2 polypeptide and wherein the plant exhibits at least one phenotype selected from the group consisting of: increased drought tolerance, increased yield, increased abiotic stress tolerance, and increased biomass compared to the control plant.

The maize plant may exhibit increased abiotic stress tolerance, and the abiotic stress may be drought stress, low nitrogen stress, or both. The plant may exhibit the phenotype of increased yield under non-stress or stress conditions. The plant may exhibit the phenotype under drought stress conditions.

The DREB2 polypeptide may comprise an amino acid sequence with at least 80% sequence identity to SEQ ID NOS: 1-11, 15, 17, 19, 21, 23, 25, and 27.

The plant may be a monocot plant such as but not limited to a maize plant.

The increase in expression of a DREB2 gene may be caused by expression using a heterologous regulatory element such as for example, a promoter or an enhancer. The increase in expression of the endogenous DREB2 gene may also be caused by a mutation in the endogenous DREB2 gene or its regulatory element, and the mutation may be caused by insertional mutagenesis including but not limited to transposon mutagenesis, or it may be caused by zinc finger nuclease, Transcription Activator-Like Effector Nuclease (TALEN), CRISPR or meganuclease.

Another embodiment is a DNA construct comprising a polynucleotide, wherein the polynucleotide is operably linked to a heterologous promoter in sense orientation, wherein the construct is effective for increasing expression of a DREB2 gene in a plant, and wherein the polynucleotide comprises: (a) the nucleotide sequence of SEQ ID NO: 13 or 14; (b) a nucleotide sequence that has at least 80% sequence identity, when compared to SEQ ID NO: 13 or 14; (c) a nucleotide sequence of at least 100 contiguous nucleotides of SEQ ID NO: 13 or 14; (d) a nucleotide sequence that can hybridize under stringent conditions with the nucleotide sequence of (a).

Another embodiment is a method of making a plant in which expression of a DREB2 gene is increased, when compared to a control plant, and wherein the plant exhibits at least one phenotype selected from the group consisting of: increased yield, increased drought tolerance, increased abiotic stress tolerance, and increased biomass, compared to the control plant, the method comprising the steps of introducing into a plant a DNA construct comprising a polynucleotide operably linked to a heterologous promoter, wherein the DNA construct is effective for increasing expression of a DREB2 gene.

Another embodiment is a method of making a plant in which expression of an endogenous DREB2 gene is increased, when compared to a control plant, and wherein the plant exhibits at least one phenotype selected from the group consisting of: increased yield, increased drought tolerance, increased abiotic stress tolerance, and increased biomass, compared to the control plant, the method comprising the steps of: (a) introducing a mutation into an endogenous DREB2 gene; and (b) detecting said mutation using the Targeted Induced Local Lesions In Genomics (TILLING) method, wherein said mutation results in reducing expression of the endogenous DREB2 gene.

Another embodiment is a method of enhancing seed yield in a plant, when compared to a control plant, wherein the plant exhibits enhanced yield under either drought stress conditions, or non-stress conditions, or both, the method comprising the step of increasing expression of the endogenous DREB2 gene in a plant.

Another embodiment is a method of making a plant in which activity of an endogenous DREB2 polypeptide is increased, when compared to the activity of wild-type DREB2 polypeptide from a control plant, and wherein the plant exhibits at least one phenotype selected from the group consisting of: increased yield, increased drought tolerance, increased abiotic stress tolerance and increased biomass, compared to the control plant, wherein the method comprises the steps of introducing into a plant a DNA construct comprising a polynucleotide operably linked to a heterologous promoter, wherein the polynucleotide encodes a fragment or a variant of a polypeptide having an amino acid sequence of at least 80% sequence identity, when compared to SEQ ID NOS: 1-11, 15, 17, 19, 21, 23, 25, and 27, wherein the fragment or the variant confers a dominant-negative phenotype in the plant.

Another embodiment is a plant comprising any of the DNA constructs disclosed herein, wherein expression of the endogenous DREB2 gene is increased in the plant, when compared to a control plant, and wherein the plant exhibits a phenotype selected from the group consisting of: increased yield, increased drought tolerance, increased abiotic stress tolerance and increased biomass, compared to the control plant. The plant may exhibit an increase in abiotic stress tolerance, and the abiotic stress may be drought stress, low nitrogen stress, or both or an increase in yield under normal growing conditions. The plant may exhibit the phenotype of increased yield and the phenotype may be exhibited under non-stress or stress conditions. The plant may be a monocot plant such as but not limited to a maize plant.

Another embodiment is a method of identifying one or more alleles associated with increased yield in a population of maize plants, the method comprising the steps of: (a) detecting in a population of maize plants one or more polymorphisms in (i) a genomic region encoding a polypeptide or (ii) a regulatory region controlling expression of the polypeptide, wherein the polypeptide comprises the amino acid sequence selected from the group consisting of SEQ ID NOS: 1-11, 15, 17, 19, 21, 23, 25, and 27, or a sequence that is 90% identical to SEQ ID NOS: 1-11, 15, 17, 19, 21, 23, 25, and 27, wherein the one or more polymorphisms in the genomic region encoding the polypeptide or in the regulatory region controlling expression of the polypeptide is associated with yield; and (b) identifying one or more alleles at the one or more polymorphisms that are associated with increased yield. The one or more alleles associated with increased yield may be used for marker assisted selection of a maize plant with increased yield. The one or more polymorphisms may be in the coding region of the polynucleotide. The regulatory region may be a promoter element.

Any progeny or seeds obtained from the plants disclosed herein are also provided herein.

### BRIEF DESCRIPTION OF THE DRAWINGS AND SEQUENCE LISTING

The disclosure can be more fully understood from the following detailed description and the accompanying drawings and Sequence Listing which form a part of this application.

FIG. 1 shows the alignment of the DREB2 polypeptides from wheat, Zea mays, Brachypodium, Sorghum, Setaria, rice, and other variants of DREB2 shown as various SEQ ID NOs. A consensus sequence is presented where a residue is shown if identical in all sequences, otherwise, a period is shown.

FIG. 2 shows phylogenetic tree for the DREB2 polypeptides shown in FIG. 1.

FIG. 3 shows the percent sequence identity values for each pair of amino acids sequences of DREB2 polypeptides displayed in FIG. 1. SEQ ID NO: 1 is the wheat DREB2 (TaDREB2) polypeptide.

Table 1 presents SEQ ID NOs for the CDS sequences of other DREB2 family members from Zea mays. The SEQ ID NOs for the corresponding amino acid sequences encoded by the cDNAs are also presented.

TABLE 1
CDS sequences Encoding maize DREB2 Polypeptides

| Plant | Clone Designation | SEQ ID NO: |
|---|---|---|
| Brachypodium | Bd_DREB3-like | 2 |
| Variant | VAR_DREB2_15 | 7 |
| Variant | VAR_DREB2_17 | 8 |
| Rice | Os_DREB2 | 3 |
| Sorghum | Sb_DREB2 | 4 |
| Maize | Zm_DREB2 | 5 |
| Setaria | Si_DREB2 | 6 |
| Variant | VAR_DREB2_19 | 9 |
| Wheat | TA_DREB2 | 1 |
| Variant | VAR_DREB2_20 | 10 |
| Variant | VAR_DREB2_21 | 11 |
| Wheat | TA_DREB2_DNA | 12 |
| Rice | OsActin Promoter | 13 |
| Maize | ZmGOS2 promoter | 14 |
| Nassella lepida | NL-DREB2-1 | 15 |
| Nassella lepida | NL-DREB2-1-CDS | 16 |
| Bromus ciliatus | BC-DREB2-2 | 17 |
| Bromus ciliatus | BC-DREB2-2-CDS | 18 |
| Deschampsia | DC-DREB2-1 | 19 |
| cespitosa | | |
| Deschampsia | DC-DREB2-1-CDS | 20 |
| cespitosa | | |
| Bromus kalmii | BK-DREB2-1 | 21 |
| Bromus kalmii | BK-DREB2-1-CDS | 22 |
| Nassella lepida | NL-DREB2-3 | 23 |
| Nassella lepida | NL-DREB2-3-CDS | 24 |
| Elymus californicus | EC-DREB2-2 | 25 |
| Elymus californicus | EC-DREB2-2-CDS | 26 |
| Achnatherum | AL-DREB2-1 | 27 |
| lettermanii | | |
| Achnatherum | AL-DREB2-1-CDS | 28 |
| lettermanii | | |

The sequence descriptions and Sequence Listing attached hereto comply with the rules governing nucleotide and/or amino acid sequence disclosures in patent applications as set forth in 37 C.F.R. § 1.821-1.825.

The Sequence Listing contains the one letter code for nucleotide sequence characters and the three letter codes for amino acids as defined in conformity with the IUPAC-IUBMB standards described in Nucleic Acids Res. 13:3021-3030 (1985) and in the Biochemical J. 219 (No. 2):345-373 (1984) which are herein incorporated by reference. The symbols and format used for nucleotide and amino acid sequence data comply with the rules set forth in 37 C.F.R. § 1.822.

### DETAILED DESCRIPTION

The disclosure of each reference set forth herein is hereby incorporated by reference in its entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

As Used Herein:

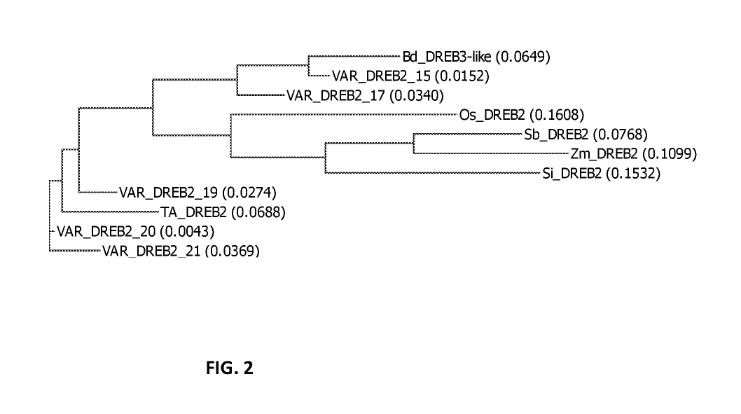

The term “DREB2 gene” refers herein to the gene that encodes for one or more of the DREB2 polypeptides disclosed herein. The term “DREB2 polypeptide” refers herein to a polypeptide that is represented by SEQ ID NO: 1 and orthologs disclosed herein (see Table 1) that are clustered with it in the clade in the phylogenetic tree shown in FIG. 2.

The term “DREB2 polypeptide” refers herein to the polypeptide given in SEQ ID NO:2 and the homologues clustered with SEQ ID NO:2 in clade 1 (FIG. 8 and Table 1 and Table 2). The terms OsDREB2, SbDREB2 and GmDREB2 refer respectively to DREB2 homologs from Oryza sativa, Sorghum bicolor and Glycine max.

The term “DREB2 polypeptide”, as referred to herein is a polypeptide comprising an amino acid sequence with at least 80% sequence identity to SEQ ID NOS: 1-11, 15, 17, 19, 21, 23, 25, and 27.

DREB2 polypeptides as referred to herein, belong to the dehydration-responsiveelement-binding (DREB) protein/C-repeat binding factors (CBFs) belong to APETALA2 (AP2) family transcription factors.

The terms “monocot” and “monocotyledonous plant” are used interchangeably herein. A monocot of the current disclosure includes the Gramineae or Poaceae.

The terms “dicot” and “dicotyledonous plant” are used interchangeably herein. A dicot of the current disclosure includes the following families: Brassicaceae, Leguminosae, and Solanaceae.

The terms “full complement” and “full-length complement” are used interchangeably herein, and refer to a complement of a given nucleotide sequence, wherein the complement and the nucleotide sequence consist of the same number of nucleotides and are 100% complementary.

An “Expressed Sequence Tag” (“EST”) is a DNA sequence derived from a cDNA library and therefore is a sequence which has been transcribed. An EST is typically obtained by a single sequencing pass of a cDNA insert. The sequence of an entire cDNA insert is termed the “Full-Insert Sequence” (“FIS”). A “Contig” sequence is a sequence assembled from two or more sequences that can be selected from, but not limited to, the group consisting of an EST, FIS and PCR sequence. A sequence encoding an entire or functional protein is termed a “Complete Gene Sequence” (“CGS”) and can be derived from an FIS or a contig.

A “trait” generally refers to a physiological, morphological, biochemical, or physical characteristic of a plant or a particular plant material or cell. In some instances, this characteristic is visible to the human eye, such as seed or plant size, or can be measured by biochemical techniques, such as detecting the protein, starch, or oil content of seed or leaves, or by observation of a metabolic or physiological process, e.g. by measuring tolerance to water deprivation or particular salt or sugar concentrations, or by the observation of the expression level of a gene or genes, or by agricultural observations such as osmotic stress tolerance or yield.

“Agronomic characteristic” is a measurable parameter including but not limited to, abiotic stress tolerance, greenness, yield, growth rate, biomass, fresh weight at maturation, dry weight at maturation, fruit yield, seed yield, total plant nitrogen content, fruit nitrogen content, seed nitrogen content, nitrogen content in a vegetative tissue, total plant free amino acid content, fruit free amino acid content, seed free amino acid content, free amino acid content in a vegetative tissue, total plant protein content, fruit protein content, seed protein content, protein content in a vegetative tissue, drought tolerance, nitrogen uptake, root lodging, harvest index, stalk lodging, plant height, ear height, ear length, salt tolerance, early seedling vigor and seedling emergence under low temperature stress.

Abiotic stress may be at least one condition selected from the group consisting of: drought, water deprivation, flood, high light intensity, high temperature, low temperature, salinity, etiolation, defoliation, heavy metal toxicity, anaerobiosis, nutrient deficiency, nutrient excess, UV irradiation, atmospheric pollution (e.g., ozone) and exposure to chemicals (e.g., paraquat) that induce production of reactive oxygen species (ROS). Nutrients include, but are not limited to, the following: nitrogen (N), phosphorus (P), potassium (K), calcium (Ca), magnesium (Mg) and sulfur (S). For example, the abiotic stress may be drought stress, low nitrogen stress, or both.

“Nitrogen limiting conditions” or “low nitrogen stress” refers to conditions where the amount of total available nitrogen (e.g., from nitrates, ammonia, or other known sources of nitrogen) is not sufficient to sustain optimal plant growth and development. One skilled in the art would recognize conditions where total available nitrogen is sufficient to sustain optimal plant growth and development. One skilled in the art would recognize what constitutes sufficient amounts of total available nitrogen, and what constitutes soils, media and fertilizer inputs for providing nitrogen to plants. Nitrogen limiting conditions will vary depending upon a number of factors, including but not limited to, the particular plant and environmental conditions.

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions.

A plant with “increased stress tolerance” can exhibit increased tolerance to one or more different stress conditions.

“Stress tolerance activity” of a polypeptide indicates that over-expression of the polypeptide in a transgenic plant confers increased stress tolerance to the transgenic plant relative to a reference or control plant.

Increased biomass can be measured, for example, as an increase in plant height, plant total leaf area, plant fresh weight, plant dry weight or plant seed yield, as compared with control plants.

The ability to increase the biomass or size of a plant would have several important commercial applications. Crop species may be generated that produce larger cultivars, generating higher yield in, for example, plants in which the vegetative portion of the plant is useful as food, biofuel or both.

Increased leaf size may be of particular interest. Increasing leaf biomass can be used to increase production of plant-derived pharmaceutical or industrial products. An increase in total plant photosynthesis is typically achieved by increasing leaf area of the plant. Additional photosynthetic capacity may be used to increase the yield derived from particular plant tissue, including the leaves, roots, fruits or seed, or permit the growth of a plant under decreased light intensity or under high light intensity.

Modification of the biomass of another tissue, such as root tissue, may be useful to improve a plant's ability to grow under harsh environmental conditions, including drought or nutrient deprivation, because larger roots may better reach water or nutrients or take up water or nutrients.

For some ornamental plants, the ability to provide larger varieties would be highly desirable. For many plants, including fruit-bearing trees, trees that are used for lumber production, or trees and shrubs that serve as view or wind screens, increased stature provides improved benefits in the forms of greater yield or improved screening.

“Nitrogen stress tolerance” is a trait of a plant and refers to the ability of the plant to survive under nitrogen limiting conditions over prolonged periods of time, without exhibiting the same degree of physiological or physical deterioration relative to the reference or control plant grown under similar stress conditions.

“Increased nitrogen stress tolerance” of a plant is measured relative to a reference or control plant, and means that the nitrogen stress tolerance of the plant is increased by any amount or measure when compared to the nitrogen stress tolerance of the reference or control plant.

A “nitrogen stress tolerant plant” is a plant that exhibits nitrogen stress tolerance. A nitrogen stress tolerant plant may be a plant that exhibits an increase in at least one agronomic characteristic relative to a control plant under nitrogen limiting conditions.

“Environmental conditions” refer to conditions under which the plant is grown, such as the availability of water, availability of nutrients (for example nitrogen), or the presence of insects or disease.

“Stay-green” or “staygreen” is a term used to describe a plant phenotype, e.g., whereby leaf senescence (most easily distinguished by yellowing of leaf associated with chlorophyll degradation) is delayed compared to a standard reference or a control. The staygreen phenotype has been used as selective criterion for the development of improved varieties of crop plants such as corn, rice and sorghum, particularly with regard to the development of stress tolerance, and yield enhancement.

“Increase in staygreen phenotype” as referred to in here, indicates retention of green leaves, delayed foliar senescence and significantly healthier canopy in a plant, compared to control plant.

The growth and emergence of maize silks play a role in the determination of yield under drought. When soil water deficit occurs before flowering, silk emergence out of the husks may be delayed while anthesis is largely unaffected, resulting in an increased anthesis-silking interval (ASI). Selection for reduced ASI has been used successfully to increase drought tolerance of maize.

Terms used herein to describe thermal time include “growing degree days” (GDD), “growing degree units” (GDU) and “heat units” (HU).

“Transgenic” generally refers to any cell, cell line, callus, tissue, plant part or plant, the genome of which has been altered by the presence of a heterologous nucleic acid, such as a DNA construct or a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, plant propagules, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

“Propagule” includes all products of meiosis and mitosis able to propagate a new plant, including but not limited to, seeds, spores and parts of a plant that serve as a means of vegetative reproduction, such as corms, tubers, offsets, or runners. Propagule also includes grafts where one portion of a plant is grafted to another portion of a different plant (even one of a different species) to create a living organism. Propagule also includes all plants and seeds produced by cloning or by bringing together meiotic products, or allowing meiotic products to come together to form an embryo or fertilized egg (naturally or with human intervention).

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a DNA construct or a recombinant DNA construct.

The commercial development of genetically improved germplasm has also advanced to the stage of introducing multiple traits into crop plants, often referred to as a gene stacking approach. In this approach, multiple genes conferring different characteristics of interest can be introduced into a plant. In case of DNA constructs, as disclosed herein, gene stacking approach may encompass expression of more than one DREB2 gene, or may also refer to stacking of a DNA construct with a recombinant DNA construct that leads to overexpression of a particular gene or polypeptide. Gene stacking can be accomplished by many means including but not limited to co-transformation, retransformation, and crossing lines with different transgenes or breeding with other drought tolerance varieties displaying non-transgenic traits, such as for example, native drought tolerance.

The DNA constructs and nucleic acid sequences of the current disclosure may be used in combination (“stacked”) with other polynucleotide sequences of interest in order to create plants with a desired phenotype. The desired combination may affect one or more traits; that is, certain combinations may be created for modulation of gene expression affecting DREB2 gene activity or expression. Other combinations may be designed to produce plants with a variety of desired traits including but not limited to increased yield and altered agronomic characteristics. “Transgenic plant” also includes reference to plants which comprise more than one heterologous polynucleotide within their genome. Each heterologous polynucleotide may confer a different trait to the transgenic plant.

In an embodiment, a DNA construct comprising a polynucleotide, wherein the polynucleotide encodes a polypeptide comprising a sequence that is at least 95% identical to SEQ ID NO: 1, is operably linked in sense orientation to a heterologous promoter, wherein the expression of the polynucleotide in a maize results in an increased yield of at least about 5% as compared to a control plant not expressing the polypeptide. In an embodiment, the yield gain is about 1%, 2%, 3%, 4%, 5%, 6%, 7%, 8%, 9% or 10% as compared to the control plant not expressing the Dreb2a gene to a level expressed by the plant in consideration.

The term “endogenous” relates to any gene or nucleic acid sequence that is already present in a cell.

“Heterologous” with respect to sequence means a sequence that originates from a foreign species, or, if from the same species, is substantially modified from its native form in composition and/or genomic locus by deliberate human intervention.

“Polynucleotide”, “nucleic acid sequence”, “nucleotide sequence”, or “nucleic acid fragment” are used interchangeably and is a polymer of RNA or DNA that is single- or double-stranded, optionally containing synthetic, non-natural or altered nucleotide bases. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

“Polypeptide”, “peptide”, “amino acid sequence” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The terms “polypeptide”, “peptide”, “amino acid sequence”, and “protein” are also inclusive of modifications including, but not limited to, glycosylation, lipid attachment, sulfation, gamma-carboxylation of glutamic acid residues, hydroxylation and ADP-ribosylation.

“Messenger RNA (mRNA)” generally refers to the RNA that is without introns and that can be translated into protein by the cell.

“cDNA” generally refers to a DNA that is complementary to and synthesized from a mRNA template using the enzyme reverse transcriptase. The cDNA can be single-stranded or converted into the double-stranded form using the Klenow fragment of DNA polymerase I.

“Coding region” generally refers to the portion of a messenger RNA (or the corresponding portion of another nucleic acid molecule such as a DNA molecule) which encodes a protein or polypeptide. “Non-coding region” generally refers to all portions of a messenger RNA or other nucleic acid molecule that are not a coding region, including but not limited to, for example, the promoter region, 5′ untranslated region (“UTR”), 3′ UTR, intron and terminator. The terms “coding region” and “coding sequence” are used interchangeably herein. The terms “non-coding region” and “non-coding sequence” are used interchangeably herein.

“Mature” protein generally refers to a post-translationally processed polypeptide; i.e., one from which any pre- or pro-peptides present in the primary translation product have been removed.

“Precursor” protein generally refers to the primary product of translation of mRNA; i.e., with pre- and pro-peptides still present. Pre- and pro-peptides may be and are not limited to intracellular localization signals.

“Isolated” generally refers to materials, such as nucleic acid molecules and/or proteins, which are substantially free or otherwise removed from components that normally accompany or interact with the materials in a naturally occurring environment. Isolated polynucleotides may be purified from a host cell in which they naturally occur. Conventional nucleic acid purification methods known to skilled artisans may be used to obtain isolated polynucleotides. The term also embraces recombinant polynucleotides and chemically synthesized polynucleotides.

As used herein the terms non-genomic nucleic acid sequence or non-genomic nucleic acid molecule generally refer to a nucleic acid molecule that has one or more change in the nucleic acid sequence compared to a native or genomic nucleic acid sequence. In some embodiments the change to a native or genomic nucleic acid molecule includes but is not limited to: changes in the nucleic acid sequence due to the degeneracy of the genetic code; codon optimization of the nucleic acid sequence for expression in plants; changes in the nucleic acid sequence to introduce at least one amino acid substitution, insertion, deletion and/or addition compared to the native or genomic sequence; removal of one or more intron associated with a genomic nucleic acid sequence; insertion of one or more heterologous introns; deletion of one or more upstream or downstream regulatory regions associated with a genomic nucleic acid sequence; insertion of one or more heterologous upstream or downstream regulatory regions; deletion of the 5′ and/or 3′ untranslated region associated with a genomic nucleic acid sequence; and insertion of a heterologous 5′ and/or 3′ untranslated region.

“Recombinant” generally refers to an artificial combination of two otherwise separated segments of sequence, e.g., by chemical synthesis or by the manipulation of isolated segments of nucleic acids by genetic engineering techniques. “Recombinant” also includes reference to a cell or vector, that has been modified by the introduction of a heterologous nucleic acid or a cell derived from a cell so modified, but does not encompass the alteration of the cell or vector by naturally occurring events (e.g., spontaneous mutation, natural transformation/transduction/transposition) such as those occurring without deliberate human intervention.

“Recombinant DNA construct” generally refers to a combination of nucleic acid fragments that are not normally found together in nature. Accordingly, a recombinant DNA construct may comprise regulatory sequences and coding sequences that are derived from different sources, or regulatory sequences and coding sequences derived from the same source, but arranged in a manner different than that normally found in nature. The terms “recombinant DNA construct” and “recombinant construct” are used interchangeably herein.

“DNA construct” is a recombinant DNA construct which when transformed or stably integrated into the genome of the plant, results in the desired expression or silencing of a gene in the plant.

The terms “reference”, “reference plant”, “control”, “control plant”, “wild-type” or “wild-type plant” are used interchangeably herein, and refers to a parent, null, or non-transgenic plant of the same species that lacks the expression of the corresponding DREB2 gene. A control plant as defined herein is a plant that is not made according to any of the methods disclosed herein. A control plant can also be a parent plant that contains a wild-type allele of a DREB2 gene. A wild-type plant would be: (1) a plant that carries the unaltered or not modulated form of a gene or allele, or (2) the starting material/plant from which the plants produced by the methods described herein are derived.

Various assays for measuring gene expression are well known in the art and can be done at the protein level (examples include, but are not limited to, Western blot, ELISA) or at the mRNA level such as by RT-PCR.

In certain aspects of the disclosure, the DNA construct is sense or antisense DNA construct.

A polynucleotide sequence is said to “encode” a sense or antisense RNA molecule, or RNA silencing or interference molecule or a polypeptide, if the polynucleotide sequence can be transcribed (in spliced or unspliced form) and/or translated into the RNA or polypeptide, or a subsequence thereof.

“Expression of a gene” or “expression of a nucleic acid” means transcription of DNA into RNA (optionally including modification of the RNA, e.g., splicing), translation of RNA into a polypeptide (possibly including subsequent modification of the polypeptide, e.g., posttranslational modification), or both transcription and translation, as might be indicated by the context.

Small RNAs play an important role in controlling gene expression. Regulation of many developmental processes, including flowering, is controlled by small RNAs. It is now possible to engineer changes in gene expression of plant genes by using transgenic constructs which produce small RNAs in the plant.

Small RNAs appear to function by base-pairing to complementary RNA or DNA target sequences. When bound to RNA, small RNAs trigger either RNA cleavage or translational inhibition of the target sequence. When bound to DNA target sequences, it is thought that small RNAs can mediate DNA methylation of the target sequence. The consequence of these events, regardless of the specific mechanism, is that gene expression is inhibited.

MicroRNAs (miRNAs) are noncoding RNAs of about 19 to about 24 nucleotides (nt) in length that have been identified in both animals and plants (Lagos-Quintana et al., Science 294:853-858 (2001), Lagos-Quintana et al., Curr. Biol. 12:735-739 (2002); Lau et al., Science 294:858-862 (2001); Lee and Ambros, Science 294:862-864 (2001); Llave et al., Plant Cell 14:1605-1619 (2002); Mourelatos et al., Genes. Dev. 16:720-728 (2002); Park et al., Curr. Biol. 12:1484-1495 (2002); Reinhart et al., Genes. Dev. 16:1616-1626 (2002)). They are processed from longer precursor transcripts that range in size from approximately 70 to 200 nt, and these precursor transcripts have the ability to form stable hairpin structures.

MicroRNAs (miRNAs) appear to regulate target genes by binding to complementary sequences located in the transcripts produced by these genes. It seems likely that miRNAs can enter at least two pathways of target gene regulation: (1) translational inhibition; and (2) RNA cleavage. MicroRNAs entering the RNA cleavage pathway are analogous to the 21-25 nt short interfering RNAs (siRNAs) generated during RNA interference (RNAi) in animals and posttranscriptional gene silencing (PTGS) in plants, and likely are incorporated into an RNA-induced silencing complex (RISC) that is similar or identical to that seen for RNAi.

Gene Disruption Techniques:

The expression or activity of the DREB2 gene and/or polypeptide can be modulated by modifying the gene encoding the DREB2 polypeptide or a regulatory element of the endogenous gene. One way of modulating a gene expression is by insertional mutagenesis. The gene can be modulated by mutagenizing the plant or plant cell using random or targeted mutagenesis.

“TILLING” or “Targeting Induced Local Lesions IN Genomics” refers to a mutagenesis technology useful to generate and/or identify, and to eventually isolate mutagenized variants of a particular nucleic acid with modulated expression and/or activity (McCallum et al., (2000), Plant Physiology 123:439-442; McCallum et al., (2000) Nature Biotechnology 18:455-457; and, Colbert et al., (2001) Plant Physiology 126:480-484).

The plant containing the mutated DREB2 gene can be crossed with other plants to introduce the mutation into another plant. This can be done using standard breeding techniques.

Homologous recombination allows introduction in a genome of a selected nucleic acid at a defined selected position. Homologous recombination has been demonstrated in plants. See, e.g., Puchta et al. (1994), Experientia 50: 277-284; Swoboda et al. (1994), EMBO J. 13: 484-489; Offringa et al. (1993), Proc. Natl. Acad. Sci. USA 90: 7346-7350; Kempin et al. (1997) Nature 389:802-803; and, Terada et al., (2002) Nature Biotechnology, 20(10):1030-1034).

Methods for performing homologous recombination in plants have been described not only for model plants (Offringa et al. (1990) EMBO J. October; 9(10):3077-84) but also for crop plants, for example rice (Terada R, Urawa H, Inagaki Y, Tsugane K, lida S. Nat Biotechnol. 2002 20(10):1030-4; lida and Terada: Curr Opin Biotechnol. 2004 April; 15(2):1328). The nucleic acid to be introduced (which may be DREB2 nucleic acid or a variant thereof) need not be targeted to the locus of the DREB2 gene, but may be introduced into, for example, regions of high expression. The nucleic acid to be introduced may be a dominant negative allele used to replace the endogenous gene or may be introduced in addition to the endogenous gene.

Another way of introducing gene disruptions into a DREB2 gene can be by introducing site-specific mutations into DREB2 genes. Mutations can be introduced in the DREB2 gene by using proteins that can introduce DNA damage into preselected regions of the plant genome. Such proteins or catalytic domains are sometimes referred to as “DNA mutator enzymes”. The DNA damage can lead to a DSB (double strand break) in double stranded DNA). The DNA mutator enzyme domain may be fused to a protein that binds to specific DNA sites.

Examples of DNA mutator enzyme domains include, but are not limited to catalytic domains such as DNA glycolases, DNA recombinase, transposase, and DNA nucleases (PCT publication No. WO2014127287; US Patent Publication No. US20140087426; incorporated herein by reference).

DNA glycolases are a family of enzymes involved in base excision repair, the mechanism by which damaged bases in DNA are removed and replaced. DNA glycolases include, but are not limited to, 3-methyladenine glycosylase (Mag1p) and uracil DNA glycolases.

DNA nuclease domains are another type of enzymes that can be used to introduce DNA damage or mutation. A DNA nuclease domain is an enzymatically active protein or fragment thereof that causes DNA cleavage resulting in a DSB.

DNA nucleases and other mutation enzyme domains may be fused with DNA binding domains to produce the DSBs in the target DNA. DNA binding domains include, for example, an array specific DNA binding domain or a site-specific DNA binding domain. Site specific DNA binding domain include but are not limited to a TAL (Transcription Activator-Like Effector) or a zinc finger binding domain.

Examples of DNA-binding domains fused to DNA nucleases include but are not limited to TALEN and multiple TALENs. Transcription Activator-Like Effector Nucleases (TALENs) are artificial restriction enzymes generated by fusing the TAL effector DNA binding domain to a DNA enzyme domain. TAL proteins are produced by bacteria and include a highly conserved 33-34 amino acid DNA binding domain sequence (PCT publication No. WO2014127287; US Patent Publication No. US20140087426).

The original TALEN chimera were prepared using the wild-type FokI endonuclease domain. However, TALEN may also include chimera made from Fok1 endonuclease domain variants with mutations designed to improve cleavage specificity and cleavage activity. In some instances multiple TALENs can be expressed to target multiple genomic regions.

A zinc finger is another type of DNA binding domain that can be used for introducing mutations into the target DNA.

Various protein engineering techniques can be used to alter the DNA-binding specificity of zinc fingers and tandem repeats of such engineered zinc fingers can be used to target desired genomic DNA sequences. Fusing a second protein domain such as a transcriptional repressor to a zinc finger that can bind near the promoter of the YEP gene can reduce the expression levels of DREB2 gene.

In one embodiment, a regulatory element driving the endogenous gene expression or the coding sequence itself, for example, may be edited or inserted into a plant by genome editing using a CRISPR/Cas9 system.

CRISPR loci (Clustered Regularly Interspaced Short Palindromic Repeats) (also known as SPIDRs—SPacer Interspersed Direct Repeats) constitute a family of recently described DNA loci. CRISPR loci consist of short and highly conserved DNA repeats (typically 24 to 40 bp, repeated from 1 to 140 times—also referred to as CRISPR-repeats) which are partially palindromic. The repeated sequences (usually specific to a species) are interspaced by variable sequences of constant length (typically 20 to 58 by depending on the CRISPR locus (WO2007/025097 published Mar. 1, 2007).

Cas gene relates to a gene that is generally coupled, associated or close to or in the vicinity of flanking CRISPR loci. The terms “Cas gene”, “CRISPR-associated (Cas) gene” are used interchangeably herein. (Haft et al. (2005) Computational Biology, PLoS Comput Biol 1(6): e60. doi:10.1371/journal.pcbi.0010060). As described therein, 41 CRISPR-associated (Cas) gene families are described, in addition to the four previously known gene families. It shows that CRISPR systems belong to different classes, with different repeat patterns, sets of genes, and species ranges. The number of Cas genes at a given CRISPR locus can vary between species.

Cas endonuclease relates to a Cas protein encoded by a Cas gene, wherein said Cas protein is capable of introducing a double strand break into a DNA target sequence. The Cas endonuclease is guided by a guide polynucleotide to recognize and optionally introduce a double strand break at a specific target site into the genome of a cell (U.S. 2015/0082478). The guide polynucleotide/Cas endonuclease system includes a complex of a Cas endonuclease and a guide polynucleotide that is capable of introducing a double strand break into a DNA target sequence. The Cas endonuclease unwinds the DNA duplex in close proximity of the genomic target site and cleaves both DNA strands upon recognition of a target sequence by a guide RNA if a correct protospacer-adjacent motif (PAM) is approximately oriented at the 3′ end of the target sequence. The Cas endonuclease can be introduced directly into a cell by any method known in the art, for example, but not limited to transient introduction methods, transfection and/or topical application.

As used herein, the term “guide RNA” relates to a synthetic fusion of two RNA molecules, a crRNA (CRISPR RNA) comprising a variable targeting domain, and a tracrRNA. In one embodiment, the guide RNA comprises a variable targeting domain of 12 to 30 nucleotide sequences and a RNA fragment that can interact with a Cas endonuclease.

As used herein, the term “guide polynucleotide”, relates to a polynucleotide sequence that can form a complex with a Cas endonuclease and enables the Cas endonuclease to recognize and optionally cleave a DNA target site (U.S. 2015/0082478). The guide polynucleotide can be a single molecule or a double molecule. The guide polynucleotide sequence can be a RNA sequence, a DNA sequence, or a combination thereof (a RNA-DNA combination sequence). Optionally, the guide polynucleotide can comprise at least one nucleotide, phosphodiester bond or linkage modification such as, but not limited, to Locked Nucleic Acid (LNA), 5-methyl dC, 2,6-Diaminopurine, 2′-Fluoro A, 2′-Fluoro U, 2′-O-Methyl RNA, phosphorothioate bond, linkage to a cholesterol molecule, linkage to a polyethylene glycol molecule, linkage to a spacer 18 (hexaethylene glycol chain) molecule, or 5′ to 3′ covalent linkage resulting in circularization. A guide polynucleotide that solely comprises ribonucleic acids is also referred to as a “guide RNA”.

The term “variable targeting domain” or “VT domain” is used interchangeably herein and includes a nucleotide sequence that is complementary to one strand (nucleotide sequence) of a double strand DNA target site. The % complementation between the first nucleotide sequence domain (VT domain) and the target sequence can be at least 50%, 51%, 52%, 53%, 54%, 55%, 56%, 57%, 58%, 59%, 60%, 61%, 62%, 63%, 63%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. The variable target domain can be at least 12, 13, 14, 15, 16, 17, 18, 19, 20, 21, 22, 23, 24, 25, 26, 27, 28, 29 or 30 nucleotides in length. In some embodiments, the variable targeting domain comprises a contiguous stretch of 12 to 30 nucleotides. The variable targeting domain can be composed of a DNA sequence, a RNA sequence, a modified DNA sequence, a modified RNA sequence, or any combination thereof.

The term “Cas endonuclease recognition domain” or “CER domain” of a guide polynucleotide is used interchangeably herein and includes a nucleotide sequence (such as a second nucleotide sequence domain of a guide polynucleotide), that interacts with a Cas endonuclease polypeptide. The CER domain can be composed of a DNA sequence, a RNA sequence, a modified DNA sequence, a modified RNA sequence (see for example modifications described herein), or any combination thereof.

The nucleotide sequence linking the crNucleotide and the tracrNucleotide of a single guide polynucleotide can comprise a RNA sequence, a DNA sequence, or a RNA-DNA combination sequence. In one embodiment, the nucleotide sequence linking the crNucleotide and the tracrNucleotide of a single guide polynucleotide can be at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 78, 79, 80, 81, 82, 83, 84, 85, 86, 87, 88, 89, 90, 91, 92, 93, 94, 95, 96, 97, 98, 99 or 100 nucleotides in length. In another embodiment, the nucleotide sequence linking the crNucleotide and the tracrNucleotide of a single guide polynucleotide can comprise a tetraloop sequence, such as, but not limiting to a GAAA tetraloop sequence.

The present disclosure encompasses variants and subsequences of the polynucleotides and polypeptides described herein.

The term “variant” with respect to a polynucleotide or DNA refers to a polynucleotide that contains changes in which one or more nucleotides of the original sequence is deleted, added, and/or substituted while substantially maintaining the function of the polynucleotide. For example, a variant of a promoter that is disclosed herein can have minor changes in its sequence without substantial alteration to its regulatory function.

The term “variant” with respect to a polypeptide refres to an amino acid sequence that is altered by one or more amino acids with respect to a reference sequence. The variant can have “conservative changes, wherein a substituted amino acid has similar structural or chemical properties, for example, and replacement of leucine with isoleucine. Alternatively, a variant can have “non-conservative” changes, for example, replacement of a glycine with a tryptophan. Analogous minor variation can also include amino acid deletion or insertion, or both.

Guidance in determining which nucleotides or amino acids for generating polynucleotide or polypeptide variants can be found using computer programs well known in the art.

The terms “fragment” and “subsequence” are used interchangeably herein, and refer to any portion of an entire sequence.

The terms “entry clone” and “entry vector” are used interchangeably herein.

“Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences. The terms “regulatory sequence” and “regulatory element” are used interchangeably herein.

“Promoter” generally refers to a nucleic acid fragment capable of controlling transcription of another nucleic acid fragment.

“Promoter functional in a plant” is a promoter capable of controlling transcription in plant cells whether or not its origin is from a plant cell.

“Tissue-specific promoter” and “tissue-preferred promoter” are used interchangeably, and refer to a promoter that is expressed predominantly but not necessarily exclusively in one tissue or organ, but that may also be expressed in one specific cell.

“Developmentally regulated promoter” generally refers to a promoter whose activity is determined by developmental events.

“Operably linked” generally refers to the association of nucleic acid fragments in a single fragment so that the function of one is regulated by the other. For example, a promoter is operably linked with a nucleic acid fragment when it is capable of regulating the transcription of that nucleic acid fragment.

“Phenotype” means the detectable characteristics of a cell or organism.

“Introduced” in the context of inserting a nucleic acid fragment (e.g., a recombinant DNA construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

A “transformed cell” is any cell into which a nucleic acid fragment (e.g., a recombinant DNA construct) has been introduced.

“Transformation” as used herein generally refers to both stable transformation and transient transformation.

“Stable transformation” generally refers to the introduction of a nucleic acid fragment into a genome of a host organism resulting in genetically stable inheritance. Once stably transformed, the nucleic acid fragment is stably integrated in the genome of the host organism and any subsequent generation.

“Transient transformation” generally refers to the introduction of a nucleic acid fragment into the nucleus, or DNA-containing organelle, of a host organism resulting in gene expression without genetically stable inheritance.

“Allele” is one of several alternative forms of a gene occupying a given locus on a chromosome. When the alleles present at a given locus on a pair of homologous chromosomes in a diploid plant are the same that plant is homozygous at that locus. If the alleles present at a given locus on a pair of homologous chromosomes in a diploid plant differ that plant is heterozygous at that locus. If a transgene is present on one of a pair of homologous chromosomes in a diploid plant that plant is hemizygous at that locus.

Allelic variants encompass Single nucleotide polymorphisms (SNPs), as well as Small Insertion/Deletion Polymorphisms (INDELs). The size of INDELs is usually less than 100 bp. SNPs and INDELs form the largest set of sequence variants in naturally occurring polymorphic strains of most organisms.

Plant breeding techniques known in the art and used in the maize plant breeding program include, but are not limited to, recurrent selection, bulk selection, mass selection, backcrossing, pedigree breeding, open pollination breeding, restriction fragment length polymorphism enhanced selection, genetic marker enhanced selection, double haploids and transformation. Often combinations of these techniques are used.

Sequence alignments and percent identity calculations may be determined using a variety of comparison methods designed to detect homologous sequences including, but not limited to, the MEGALIGN® program of the LASERGENE® bioinformatics computing suite (DNASTAR® Inc., Madison, Wis.). Unless stated otherwise, multiple alignment of the sequences provided herein were performed using the Clustal V method of alignment (Higgins and Sharp (1989) CABIOS. 5:151-153) with the default parameters (GAP PENALTY=10, GAP LENGTH PENALTY=10). Default parameters for pairwise alignments and calculation of percent identity of protein sequences using the Clustal V method are KTUPLE=1, GAP PENALTY=3, WINDOW=5 and DIAGONALS SAVED=5. For nucleic acids these parameters are KTUPLE=2, GAP PENALTY=5, WINDOW=4 and DIAGONALS SAVED=4. After alignment of the sequences, using the Clustal V program, it is possible to obtain “percent identity” and “divergence” values by viewing the “sequence distances” table on the same program; unless stated otherwise, percent identities and divergences provided and claimed herein were calculated in this manner.

Alternatively, the Clustal W method of alignment may be used. The Clustal W method of alignment (described by Higgins and Sharp, CABIOS. 5:151-153 (1989); Higgins, D. G. et al., Comput. Appl. Biosci. 8:189-191 (1992)) can be found in the MegAlign™ v6.1 program of the LASERGENE® bioinformatics computing suite (DNASTAR® Inc., Madison, Wis.). Default parameters for multiple alignment correspond to GAP PENALTY=10, GAP LENGTH PENALTY=0.2, Delay Divergent Sequences=30%, DNA Transition Weight=0.5, Protein Weight Matrix=Gonnet Series, DNA Weight Matrix=IUB. For pairwise alignments the default parameters are Alignment=Slow-Accurate, Gap Penalty=10.0, Gap Length=0.10, Protein Weight Matrix=Gonnet 250 and DNA Weight Matrix=IUB. After alignment of the sequences using the Clustal W program, it is possible to obtain “percent identity” and “divergence” values by viewing the “sequence distances” table in the same program.

Standard recombinant DNA and molecular cloning techniques used herein are well known in the art and are described more fully in Sambrook, J., Fritsch, E. F. and Maniatis, T. Molecular Cloning: A Laboratory Manual; Cold Spring Harbor Laboratory Press: Cold Spring Harbor, 1989 (hereinafter “Sambrook”).

Turning Now to the Embodiments:

Embodiments include isolated polynucleotides and polypeptides, recombinant DNA constructs useful for conferring drought tolerance, compositions (such as plants or seeds) comprising these recombinant DNA constructs, and methods utilizing these recombinant DNA constructs.

In one embodiment, a plant in which expression of a DREB2 gene is increased, when compared to a control plant, wherein the DREB2 gene encodes a DREB2 polypeptide and wherein the plant exhibits at least one phenotype selected from the group consisting of: increased yield, increased abiotic stress tolerance, and increased biomass compared to the control plant.

In one embodiment, a plant in which activity of a DREB2 polypeptide is increased, when compared to the activity of wild-type DREB2 polypeptide in a control plant, wherein the plant exhibits at least one phenotype selected from the group consisting of: increased yield, increased abiotic stress tolerance, and increased biomass compared to the control plant.

In one embodiment, the plant exhibits increased abiotic stress tolerance, and the abiotic stress is drought stress, low nitrogen stress, or both. In one embodiment, the plant exhibits the phenotype of increased yield and the phenotype is exhibited under non-stress conditions. In one embodiment, the plant exhibits the phenotype of increased yield and the phenotype is exhibited under stress conditions. In one embodiment, the plant exhibits the phenotype under drought stress conditions.

In one embodiment, the plant is a monocot plant. In another embodiment, the plant is a maize plant.

In one embodiment, the mutation in the endogenous DREB2 gene is caused by zinc finger nuclease, Transcription Activator-Like Effector Nuclease (TALEN), CRISPR or meganuclease.

Isolated Polynucleotides and Polypeptides:

The present disclosure includes the following isolated polynucleotides and polypeptides:

An isolated polynucleotide comprising: (i) a nucleic acid sequence encoding a DREB2 polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NOS: 1-11, 15, 17, 19, 21, 23, 25, and 27, and combinations thereof; or (ii) a full complement of the nucleic acid sequence of (i), wherein the full complement and the nucleic acid sequence of (i) consist of the same number of nucleotides and are 100% complementary. Any of the foregoing isolated polynucleotides or a fragment or subsequence of the isolated polynucleotides may be utilized in any DNA constructs of the present disclosure.

An isolated polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NOS: 1-11, 15, 17, 19, 21, 23, 25, and 27, and combinations thereof. The polypeptide is preferably a DREB2 polypeptide.

An isolated polynucleotide comprising (i) a nucleic acid sequence of at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NO: 12, and combinations thereof; (ii) a full complement of the nucleic acid sequence of (i); or (iii) a fragment or subsequence of the nucleic acid sequence of (i). Any of the foregoing isolated polynucleotides or a fragment of the isolated polynucleotides may be utilized in any DNA construct of the present disclosure. The isolated polynucleotide preferably encodes a DREB2 polypeptide.

An isolated polynucleotide comprising a nucleotide sequence, wherein the nucleotide sequence is hybridizable under stringent conditions with a DNA molecule comprising the full complement of SEQ ID NO: 12, or a subsequence thereof. The isolated polynucleotide preferably encodes a DREB2 polypeptide.

An isolated polynucleotide comprising a nucleotide sequence, wherein the nucleotide sequence is derived from SEQ ID NO: 12 by alteration of one or more nucleotides by at least one method selected from the group consisting of: deletion, substitution, addition and insertion. The isolated polynucleotide preferably encodes a DREB2 polypeptide. An isolated polynucleotide comprising a nucleotide sequence, wherein the nucleotide sequence corresponds to an allele of SEQ ID NO: 12.

It is understood, as those skilled in the art will appreciate, that the disclosure encompasses more than the specific exemplary sequences. Alterations in a nucleic acid fragment which result in the production of a chemically equivalent amino acid at a given site, but do not affect the functional properties of the encoded polypeptide, are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

The protein of the current disclosure may also be a protein which comprises an amino acid sequence comprising deletion, substitution, insertion and/or addition of one or more amino acids in an amino acid sequence presented in SEQ ID NOS: 1-11, 15, 17, 19, 21, 23, 25, and 27. The substitution may be conservative, which means the replacement of a certain amino acid residue by another residue having similar physical and chemical characteristics. Non-limiting examples of conservative substitution include replacement between aliphatic group-containing amino acid residues such as Ile, Val, Leu or Ala, and replacement between polar residues such as Lys-Arg, Glu-Asp or Gln-Asn replacement.

Proteins derived by amino acid deletion, substitution, insertion and/or addition can be prepared when DNAs encoding their wild-type proteins are subjected to, for example, well-known site-directed mutagenesis (see, e.g., Nucleic Acid Research, Vol. 10, No. 20, p. 6487-6500, 1982, which is hereby incorporated by reference in its entirety). As used herein, the term “one or more amino acids” is intended to mean a possible number of amino acids which may be deleted, substituted, inserted and/or added by site-directed mutagenesis.

Site-directed mutagenesis may be accomplished, for example, as follows using a synthetic oligonucleotide primer that is complementary to single-stranded phage DNA to be mutated, except for having a specific mismatch (i.e., a desired mutation). Namely, the above synthetic oligonucleotide is used as a primer to cause synthesis of a complementary strand by phages, and the resulting duplex DNA is then used to transform host cells. The transformed bacterial culture is plated on agar, whereby plaques are allowed to form from phage-containing single cells. As a result, in theory, 50% of new colonies contain phages with the mutation as a single strand, while the remaining 50% have the original sequence. At a temperature which allows hybridization with DNA completely identical to one having the above desired mutation, but not with DNA having the original strand, the resulting plaques are allowed to hybridize with a synthetic probe labeled by kinase treatment. Subsequently, plaques hybridized with the probe are picked up and cultured for collection of their DNA.

Techniques for allowing deletion, substitution, insertion and/or addition of one or more amino acids in the amino acid sequences of biologically active peptides such as enzymes while retaining their activity include site-directed mutagenesis mentioned above, as well as other techniques such as those for treating a gene with a mutagen, and those in which a gene is selectively cleaved to remove, substitute, insert or add a selected nucleotide or nucleotides, and then ligated.

The protein of the present disclosure may also be a protein which is encoded by a nucleic acid comprising a nucleotide sequence comprising deletion, substitution, insertion and/or addition of one or more nucleotides in the nucleotide sequence of SEQ ID NO: 12. Nucleotide deletion, substitution, insertion and/or addition may be accomplished by site-directed mutagenesis or other techniques as mentioned above.

The protein of the present disclosure may also be a protein which is encoded by a nucleic acid comprising a nucleotide sequence hybridizable under stringent conditions with the complementary strand of the nucleotide sequence of SEQ ID NO: 12.

The term “under stringent conditions” means that two sequences hybridize under moderately or highly stringent conditions. More specifically, moderately stringent conditions can be readily determined by those having ordinary skill in the art, e.g., depending on the length of DNA. The basic conditions are set forth by Sambrook et al., Molecular Cloning: A Laboratory Manual, third edition, chapters 6 and 7, Cold Spring Harbor Laboratory Press, 2001 and include the use of a prewashing solution for nitrocellulose filters 5×SSC, 0.5% SDS, 1.0 mM EDTA (pH 8.0), hybridization conditions of about 50% formamide, 2×SSC to 6×SSC at about 40-50° C. (or other similar hybridization solutions, such as Stark's solution, in about 50% formamide at about 42° C.) and washing conditions of, for example, about 40-60° C., 0.5-6×SSC, 0.1% SDS. Preferably, moderately stringent conditions include hybridization (and washing) at about 50° C. and 6×SSC. Highly stringent conditions can also be readily determined by those skilled in the art, e.g., depending on the length of DNA.

Generally, such conditions include hybridization and/or washing at higher temperature and/or lower salt concentration (such as hybridization at about 65° C., 6×SSC to 0.2×SSC, preferably 6×SSC, more preferably 2×SSC, most preferably 0.2×SSC), compared to the moderately stringent conditions. For example, highly stringent conditions may include hybridization as defined above, and washing at approximately 65-68° C., 0.2×SSC, 0.1% SDS. SSPE (1×SSPE is 0.15 M NaCl, 10 mM NaH2PO4, and 1.25 mM EDTA, pH 7.4) can be substituted for SSC (1×SSC is 0.15 M NaCl and 15 mM sodium citrate) in the hybridization and washing buffers; washing is performed for 15 minutes after hybridization is completed.

It is also possible to use a commercially available hybridization kit which uses no radioactive substance as a probe. Specific examples include hybridization with an ECL direct labeling & detection system (Amersham). Stringent conditions include, for example, hybridization at 42° C. for 4 hours using the hybridization buffer included in the kit, which is supplemented with 5% (w/v) Blocking reagent and 0.5 M NaCl, and washing twice in 0.4% SDS, 0.5×SSC at 55° C. for 20 minutes and once in 2×SSC at room temperature for 5 minutes.

Recombinant DNA Constructs and DNA Constructs:

In one aspect, the present disclosure includes DNA constructs.

One embodiment is a DNA construct comprising a polynucleotide, wherein the polynucleotide is operably linked to a heterologous promoter in sense or antisense orientation, or both, wherein the construct is effective for reducing expression of an endogenous DREB2 gene in a plant, and wherein the polynucleotide comprises: (a) the nucleotide sequence of SEQ ID NO: 12; (b) a nucleotide sequence that has at least 80% sequence identity, when compared to SEQ ID NO: 12; (c) a nucleotide sequence of at least 100 contiguous nucleotides of SEQ ID NO: 12; (d) a nucleotide sequence that can hybridize under stringent conditions with the nucleotide sequence of (a); or (e) a modified plant miRNA precursor, wherein the precursor has been modified to replace the miRNA encoding region with a sequence designed to produce a miRNA directed to SEQ ID NO: 12.

In one embodiment, the DREB2 polypeptide may be from Zea mays, Glycine max, Oryza sativa, Sorghum bicolor, Saccharum officinarum, or Triticum aestivum.

In one embodiment, the promoter may be a constitutive promoter, an inducible promoter, a tissue-specific promoter.

Regulatory Sequences:

A recombinant DNA construct (including a DNA construct) of the present disclosure may comprise at least one regulatory sequence.

A regulatory sequence may be a promoter.

A number of promoters can be used in recombinant DNA constructs of the present disclosure. The promoters can be selected based on the desired outcome, and may include constitutive, tissue-specific, inducible, or other promoters for expression in the host organism.

Promoters that cause a gene to be expressed in most cell types at most times are commonly referred to as “constitutive promoters”.

High level, constitutive expression of the candidate gene under control of the 35S or UBI promoter may have pleiotropic effects, although candidate gene efficacy may be estimated when driven by a constitutive promoter. Use of tissue-specific and/or stress-specific promoters may eliminate undesirable effects but retain the ability to enhance stress tolerance. This effect has been observed in Arabidopsis (Kasuga et al. (1999) Nature Biotechnol. 17:287-91).

Suitable constitutive promoters for use in a plant host cell include, for example, the core promoter of the Rsyn7 promoter and other constitutive promoters disclosed in WO 99/43838 and U.S. Pat. No. 6,072,050; the core CaMV 35S promoter (Odell et al., Nature 313:810-812 (1985)); rice actin (McElroy et al., Plant Cell 2:163-171 (1990)); ubiquitin (Christensen et al., Plant Mol. Biol. 12:619-632 (1989) and Christensen et al., Plant Mol. Biol. 18:675-689 (1992)); pEMU (Last et al., Theor. Appl. Genet. 81:581-588 (1991)); MAS (Velten et al., EMBO J. 3:2723-2730 (1984)); ALS promoter (U.S. Pat. No. 5,659,026), the constitutive synthetic core promoter SCP1 (International Publication No. 03/033651) and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142; and 6,177,611.

In choosing a promoter to use in the methods of the disclosure, it may be desirable to use a tissue-specific or developmentally regulated promoter.

A tissue-specific or developmentally regulated promoter is a DNA sequence which regulates the expression of a DNA sequence selectively in the cells/tissues of a plant critical to tassel development, seed set, or both, and limits the expression of such a DNA sequence to the period of tassel development or seed maturation in the plant. Any identifiable promoter may be used in the methods of the present disclosure which causes the desired temporal and spatial expression.

Promoters which are seed or embryo-specific and may be useful include soybean Kunitz trypsin inhibitor (Kti3, Jofuku and Goldberg, Plant Cell 1:1079-1093 (1989)), patatin (potato tubers) (Rocha-Sosa, M., et al. (1989) EMBO J. 8:23-29), convicilin, vicilin, and legumin (pea cotyledons) (Rerie, W. G., et al. (1991) Mol. Gen. Genet. 259:149-157; Newbigin, E. J., et al. (1990) Planta 180:461-470; Higgins, T. J. V., et al. (1988) Plant. Mol. Biol. 11:683-695), zein (maize endosperm) (Schemthaner, J. P., et al. (1988) EMBO J. 7:1249-1255), phaseolin (bean cotyledon) (Segupta-Gopalan, C., et al. (1985) Proc. Natl. Acad. Sci. U.S.A. 82:3320-3324), phytohemagglutinin (bean cotyledon) (Voelker, T. et al. (1987) EMBO J. 6:3571-3577), B-conglycinin and glycinin (soybean cotyledon) (Chen, Z-L, et al. (1988) EMBO J. 7:297-302), glutelin (rice endosperm), hordein (barley endosperm) (Marris, C., et al. (1988) Plant Mol. Biol. 10:359-366), glutenin and gliadin (wheat endosperm) (Colot, V., et al. (1987) EMBO J. 6:3559-3564), and sporamin (sweet potato tuberous root) (Hattori, T., et al. (1990) Plant Mol. Biol. 14:595-604). Promoters of seed-specific genes operably linked to heterologous coding regions in chimeric gene constructions maintain their temporal and spatial expression pattern in transgenic plants. Such examples include Arabidopsis thaliana 2S seed storage protein gene promoter to express enkephalin peptides in Arabidopsis and Brassica napus seeds (Vanderkerckhove et al., Bio/Technology 7:L929-932 (1989)), bean lectin and bean beta-phaseolin promoters to express luciferase (Riggs et al., Plant Sci. 63:47-57 (1989)), and wheat glutenin promoters to express chloramphenicol acetyl transferase (Colot et al., EMBO J 6:3559-3564 (1987)). Endosperm preferred promoters include those described in e.g., U.S. Pat. No. 8,466,342; U.S. Pat. No. 7,897,841; and U.S. Pat. No. 7,847,160.

Inducible promoters selectively express an operably linked DNA sequence in response to the presence of an endogenous or exogenous stimulus, for example by chemical compounds (chemical inducers) or in response to environmental, hormonal, chemical, and/or developmental signals. Inducible or regulated promoters include, for example, promoters regulated by light, heat, stress, flooding or drought, phytohormones, wounding, or chemicals such as ethanol, jasmonate, salicylic acid, or safeners.

Promoters for use include the following: 1) the stress-inducible RD29A promoter (Kasuga et al. (1999) Nature Biotechnol. 17:287-91); 2) the barley promoter, B22E; expression of B22E is specific to the pedicel in developing maize kernels (“Primary Structure of a Novel Barley Gene Differentially Expressed in Immature Aleurone Layers”. Klemsdal, S. S. et al., Mol. Gen. Genet. 228(1/2):9-16 (1991)); and 3) maize promoter, Zag2 (“Identification and molecular characterization of ZAG1, the maize homolog of the Arabidopsis floral homeotic gene AGAMOUS”, Schmidt, R. J. et al., Plant Cell 5(7):729-737 (1993); “Structural characterization, chromosomal localization and phylogenetic evaluation of two pairs of AGAMOUS-like MADS-box genes from maize”, Theissen et al. Gene 156(2):155-166 (1995); NCBI GenBank Accession No. X80206)). Zag2 transcripts can be detected 5 days prior to pollination to 7 to 8 days after pollination (“DAP”), and directs expression in the carpel of developing female inflorescences and CimI which is specific to the nucleus of developing maize kernels. CimI transcript is detected 4 to 5 days before pollination to 6 to 8 DAP. Other useful promoters include any promoter which can be derived from a gene whose expression is maternally associated with developing female florets. Promoters for use also include the following: Zm-GOS2 (maize promoter for “Gene from Oryza sativa” (see e.g., U.S. Pat. No. 6,504,083 B1), US publication number US2012/0110700 for Sb-RCC (Sorghum promoter for Root Cortical Cell delineating protein, root specific expression), Zm-ADF4 (U.S. Pat. No. 7,902,428; Maize promoter for Actin Depolymerizing Factor), Zm-FTM1 (U.S. Pat. No. 7,842,851; maize promoter for Floral transition MADSs) promoters; OsActin promoter (WO2014160304—SEQ ID NO: 4).

Additional promoters for regulating the expression of the nucleotide sequences in plants are stalk-specific promoters. Such stalk-specific promoters include the alfalfa S2A promoter (GenBank Accession No. EF030816; Abrahams et al., Plant Mol. Biol. 27:513-528 (1995)) and S2B promoter (GenBank Accession No. EF030817) and the like, herein incorporated by reference.

Promoters may be derived in their entirety from a native gene, or be composed of different elements derived from different promoters found in nature, or even comprise synthetic DNA segments.

In one embodiment the at least one regulatory element may be an endogenous promoter operably linked to at least one enhancer element; e.g., a 35S, nos or ocs enhancer element.

Promoters for use may include: RIP2, mLIP15, ZmCOR1, Rab17, CaMV 35S, RD29A, B22E, Zag2, SAM synthetase, ubiquitin, CaMV 19S, nos, Adh, sucrose synthase, R-allele, the vascular tissue preferred promoters S2A (Genbank accession number EF030816) and S2B (Genbank accession number EF030817), and the constitutive promoter GOS2 from Zea mays. Other promoters include root preferred promoters, such as the maize NAS2 promoter, the maize Cyclo promoter (US 2006/0156439, published Jul. 13, 2006), the maize ROOTMET2 promoter (WO05063998, published Jul. 14, 2005), the CR1 BIO promoter (WO06055487, published May 26, 2006), the CRWAQ81 (WO05035770, published Apr. 21, 2005) and the maize ZRP2.47 promoter (NCBI accession number: U38790; GI No. 1063664),

DNA constructs of the present disclosure may also include other regulatory sequences, including but not limited to, translation leader sequences, introns, and polyadenylation recognition sequences. In another embodiment of the present disclosure, a recombinant DNA construct of the present disclosure further comprises an enhancer or silencer.

The promoters disclosed herein may be used with their own introns, or with any heterologous introns to drive expression of the transgene.

An intron sequence can be added to the 5′ untranslated region, the protein-coding region or the 3′ untranslated region to increase the amount of the mature message that accumulates in the cytosol. Inclusion of a spliceable intron in the transcription unit in both plant and animal expression constructs has been shown to increase gene expression at both the mRNA and protein levels up to 1000-fold. Buchman and Berg, Mol. Cell Biol. 8:4395-4405 (1988); Callis et al., Genes Dev. 1:1183-1200 (1987).

“Transcription terminator”, “termination sequences”, or “terminator” refer to DNA sequences located downstream of a protein-coding sequence, including polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht, I. L., et al., Plant Cell 1:671-680 (1989). A polynucleotide sequence with “terminator activity” generally refers to a polynucleotide sequence that, when operably linked to the 3′ end of a second polynucleotide sequence that is to be expressed, is capable of terminating transcription from the second polynucleotide sequence and facilitating efficient 3′ end processing of the messenger RNA resulting in addition of poly A tail. Transcription termination is the process by which RNA synthesis by RNA polymerase is stopped and both the processed messenger RNA and the enzyme are released from the DNA template.

Improper termination of an RNA transcript can affect the stability of the RNA, and hence can affect protein expression. Variability of transgene expression is sometimes attributed to variability of termination efficiency (Bieri et al (2002) Molecular Breeding 10: 107-117).

Examples of terminators for use include, but are not limited to, PinII terminator, SB-GKAF terminator (U.S. Appln. No. 61/514,055), Actin terminator, Os-Actin terminator, Ubi terminator, Sb-Ubi terminator, Os-Ubi terminator.

A composition of the present disclosure is a plant comprising in its genome any of the DNA constructs of the present disclosure (such as any of the constructs discussed above). Compositions also include any progeny of the plant, and any seed obtained from the plant or its progeny, wherein the progeny or seed comprises within its genome the DNA construct. Progeny includes subsequent generations obtained by self-pollination or out-crossing of a plant. Progeny also includes hybrids and inbreds.

In hybrid seed propagated crops, mature transgenic plants can be self-pollinated to produce a homozygous inbred plant. The inbred plant produces seed containing the newly introduced DNA construct. These seeds can be grown to produce plants that would exhibit an altered agronomic characteristic (e.g., an increased agronomic characteristic optionally under stress conditions), or used in a breeding program to produce hybrid seed, which can be grown to produce plants that would exhibit such an altered agronomic characteristic. The seeds may be maize seeds. The stress condition may be selected from the group of drought stress, and nitrogen stress.

The plant may be a monocotyledonous or dicotyledonous plant, for example, a maize or soybean plant. The plant may also be sunflower, sorghum, canola, wheat, alfalfa, cotton, rice, barley, millet, sugar cane or switchgrass. The plant may be a hybrid plant or an inbred plant.

In any of the embodiments described herein, the plant may exhibit less yield loss relative to the control plants, for example, at least 25%, at least 20%, at least 15%, at least 10% or at least 5% less yield loss, under water limiting conditions, or would have increased yield, for example, at least 5%, at least 10%, at least 15%, at least 20% or at least 25% increased yield, relative to the control plants under water non-limiting conditions.

In any of the embodiments described herein, the plant may exhibit less yield loss relative to the control plants, for example, at least 25%, at least 20%, at least 15%, at least 10% or at least 5% less yield loss, under stress conditions. The stress may be either drought stress, low nitrogen stress, or both.

In one embodiment, the plant may exhibit increased yield, for example, at least 5%, at least 10%, at least 15%, at least 20% or at least 25% increased yield, relative to the control plants under non-stress conditions.

Yield analysis can be done to determine whether plants that have expression levels of at least one of the DREB2 genes have an improvement in yield performance under non-stress or stress conditions, when compared to the control plants that have wild-type expression levels and activity levels of the YEP gene and polypeptide, respectively. Stress conditions can be water-limiting conditions, or low nitrogen conditions. Specifically, drought conditions or nitrogen limiting conditions can be imposed during the flowering and/or grain fill period for plants that contain the DNA construct and the control plants.

In one embodiment, the plant may exhibit phenotype, or an increase in biomass, relative to the control plants under non-stress conditions.

In one embodiment, the plant may exhibit phenotype, or an increase in biomass, relative to the control plants under stress conditions.

In one embodiment, yield can be measured in many ways, including, for example, test weight, seed weight, seed number per plant, seed number per unit area (i.e. seeds, or weight of seeds, per acre), bushels per acre, tonnes per acre, tons per acre, kilo per hectare.

The terms “stress tolerance” or “stress resistance” as used herein generally refers to a measure of a plants ability to grow under stress conditions that would detrimentally affect the growth, vigor, yield, and size, of a “non-tolerant” plant of the same species. Stress tolerant plants grow better under conditions of stress than non-stress tolerant plants of the same species. For example, a plant with increased growth rate, compared to a plant of the same species and/or variety, when subjected to stress conditions that detrimentally affect the growth of another plant of the same species would be said to be stress tolerant. A plant with “increased stress tolerance” can exhibit increased tolerance to one or more different stress conditions.

“Increased stress tolerance” of a plant is measured relative to a reference or control plant, and is a trait of the plant to survive under stress conditions over prolonged periods of time, without exhibiting the same degree of physiological or physical deterioration relative to the reference or control plant grown under similar stress conditions. Typically, when a transgenic plant comprising a recombinant DNA construct or DNA construct in its genome exhibits increased stress tolerance relative to a reference or control plant, the reference or control plant does not comprise in its genome the recombinant DNA construct or DNA construct.

“Drought” generally refers to a decrease in water availability to a plant that, especially when prolonged, can cause damage to the plant or prevent its successful growth (e.g., limiting plant growth or seed yield). “Water limiting conditions” generally refers to a plant growth environment where the amount of water is not sufficient to sustain optimal plant growth and development. The terms “drought” and “water limiting conditions” are used interchangeably herein.

“Drought tolerance” is a trait of a plant to survive under drought conditions over prolonged periods of time without exhibiting substantial physiological or physical deterioration.

“Drought tolerance activity” of a polypeptide indicates that over-expression of the polypeptide in a transgenic plant confers increased drought tolerance to the transgenic plant relative to a reference or control plant.

“Increased drought tolerance” of a plant is measured relative to a reference or control plant, and is a trait of the plant to survive under drought conditions over prolonged periods of time, without exhibiting the same degree of physiological or physical deterioration relative to the reference or control plant grown under similar drought conditions. Typically, when a transgenic plant comprising a recombinant DNA construct or DNA construct in its genome exhibits increased drought tolerance relative to a reference or control plant, the reference or control plant does not comprise in its genome the recombinant DNA construct or DNA construct.

When a transgenic plant comprising a DNA construct in its genome exhibits increased stress tolerance relative to a reference or control plant, the reference or control plant does not comprise in its genome the DNA construct.

The range of stress and stress response depends on the different plants which are used, i.e., it varies for example between a plant such as wheat and a plant such as Arabidopsis.

One of ordinary skill in the art is familiar with protocols for simulating drought conditions and for evaluating drought tolerance of plants that have been subjected to simulated or naturally-occurring drought conditions. For example, one can simulate drought conditions by giving plants less water than normally required or no water over a period of time, and one can evaluate drought tolerance by looking for differences in physiological and/or physical condition, including (but not limited to) vigor, growth, size, or root length, or in particular, leaf color or leaf area size. Other techniques for evaluating drought tolerance include measuring chlorophyll fluorescence, photosynthetic rates and gas exchange rates.

A drought stress experiment may involve a chronic stress (i.e., slow dry down) and/or may involve two acute stresses (i.e., abrupt removal of water) separated by a day or two of recovery. Chronic stress may last 8-10 days. Acute stress may last 3-5 days. The following variables may be measured during drought stress and well watered treatments of transgenic plants and relevant control plants:

The variable “% area chg_start chronic−acute2” is a measure of the percent change in total area determined by remote visible spectrum imaging between the first day of chronic stress and the day of the second acute stress.

The variable “% area chg_start chronic−end chronic” is a measure of the percent change in total area determined by remote visible spectrum imaging between the first day of chronic stress and the last day of chronic stress.

The variable “% area chg_start chronic−harvest” is a measure of the percent change in total area determined by remote visible spectrum imaging between the first day of chronic stress and the day of harvest.

The variable “% area chg_start chronic−recovery24 hr” is a measure of the percent change in total area determined by remote visible spectrum imaging between the first day of chronic stress and 24 hrs into the recovery (24 hrs after acute stress 2).

The variable “psii_acute1” is a measure of Photosystem II (PSII) efficiency at the end of the first acute stress period. It provides an estimate of the efficiency at which light is absorbed by PSII antennae and is directly related to carbon dioxide assimilation within the leaf.

The variable “psii_acute2” is a measure of Photosystem II (PSII) efficiency at the end of the second acute stress period. It provides an estimate of the efficiency at which light is absorbed by PSII antennae and is directly related to carbon dioxide assimilation within the leaf.

The variable “fv/fm_acute1” is a measure of the optimum quantum yield (Fv/Fm) at the end of the first acute stress−(variable fluorescence difference between the maximum and minimum fluorescence/maximum fluorescence)

The variable “fv/fm_acute2” is a measure of the optimum quantum yield (Fv/Fm) at the end of the second acute stress−(variable flourescence difference between the maximum and minimum fluorescence/maximum fluorescence).

The variable “leaf rolling_harvest” is a measure of the ratio of top image to side image on the day of harvest.

The variable “leaf rolling_recovery24 hr” is a measure of the ratio of top image to side image 24 hours into the recovery.

The variable “Specific Growth Rate (SGR)” represents the change in total plant surface area (as measured by a commercially available imaging instrument) over a single day (Y(t)Y0*e^{r*t}). Y(t)=Y0*e^{r*t} is equivalent to % change in Y/Δ t where the individual terms are as follows: Y(t)=Total surface area at t; Y0=Initial total surface area (estimated); r=Specific Growth Rate day⁻¹, and t=Days After Planting (“DAP”).

The variable “shoot dry weight” is a measure of the shoot weight 96 hours after being placed into a 104° C. oven.

The variable “shoot fresh weight” is a measure of the shoot weight immediately after being cut from the plant.

The Examples below describe some representative protocols and techniques for simulating drought conditions and/or evaluating drought tolerance.

One can also evaluate drought tolerance by the ability of a plant to maintain sufficient yield (at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% yield) in field testing under simulated or naturally-occurring drought conditions (e.g., by measuring for substantially equivalent yield under drought conditions compared to non-drought conditions, or by measuring for less yield loss under drought conditions compared to a control or reference plant).

A method of enhancing seed yield in a plant, when compared to a control plant, wherein the plant exhibits enhanced yield under either stress conditions, or non-stress conditions, or both, the method comprising the step of increasing expression of the endogenous DREB2 gene in a plant.

A method of making a plant in which expression of an endogenous DREB2 gene is increased, when compared to a control plant, and wherein the plant exhibits at least one phenotype selected from the group consisting of: increased yield, increased abiotic stress tolerance such as increased drought tolerance, and increased biomass, compared to the control plant, the method comprising the step of utilizing a transposon to introduce an insertion into an endogenous DREB2 gene in a plant, wherein the insertion is effective for reducing expression of an endogenous DREB2 gene.

A method of making a plant in which activity of an endogenous DREB2 polypeptide is increased, when compared to the activity of wild-type DREB2 polypeptide from a control plant, and wherein the plant exhibits at least one phenotype selected from the group consisting of: increased yield, increased abiotic stress tolerance such as increased drought tolerance and increased biomass, compared to the control plant, wherein the method comprises the steps of introducing into a plant a DNA construct comprising a polynucleotide operably linked to a heterologous promoter, wherein the polynucleotide encodes a fragment or a variant of a polypeptide having an amino acid sequence of at least 80%, 81%, 82%, 83%, 84%, 85%, 86%, 87%, 88%, 89%, 90%, 91%, 92%, 93%, 94%, 95%, 96%, 97%, 98%, 99% or 100% sequence identity, when compared to SEQ ID NOS: 1-11, 15, 17, 19, 21, 23, 25, and 27, wherein the fragment or the variant confers a dominant-negative phenotype in the plant.

A method of selecting for (or identifying) increased stress tolerance in a plant, wherein the stress is selected from the group consisting of drought stress, triple stress and osmotic stress the method comprising: (a) obtaining a transgenic plant, wherein the transgenic plant comprises in its genome a DNA construct comprising a polynucleotide operably linked to at least one regulatory element, wherein said polynucleotide comprises a nucleotide sequence, wherein the nucleotide sequence is: (i) hybridizable under stringent conditions with a DNA molecule comprising the full complement of SEQ ID NO: 12; or (ii) derived from SEQ ID NO: 12 by alteration of one or more nucleotides by at least one method selected from the group consisting of: deletion, substitution, addition and insertion; (b) obtaining a progeny plant derived from said transgenic plant, wherein the progeny plant comprises in its genome the DNA construct; and (c) selecting (or identifying) the progeny plant with increased drought tolerance, when compared to a control plant not comprising the DNA construct.

A method of selecting for (or identifying) an alteration of an agronomic characteristic in a plant, comprising (a) obtaining a transgenic plant, wherein the transgenic plant comprises in its genome a DNA construct comprising a polynucleotide operably linked to at least one regulatory sequence (for example, a promoter functional in a plant), wherein said polynucleotide encodes a polypeptide having an amino acid sequence of at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity, based on the Clustal V or Clustal W method of alignment, when compared to SEQ ID NOS: 1-11, 15, 17, 19, 21, 23, 25, and 27; (b) obtaining a progeny plant derived from said transgenic plant, wherein the progeny plant comprises in its genome the DNA construct; and (c) selecting (or identifying) the progeny plant that exhibits an alteration in at least one agronomic characteristic when compared, optionally under at least one stress condition, to a control plant not comprising the DNA construct. The at least one stress condition may be selected from the group of drought stress, and low nitrogen stress. The polynucleotide preferably encodes a DREB2 polypeptide.

The development or regeneration of plants containing the foreign, exogenous isolated nucleic acid fragment that encodes a protein of interest is well known in the art. The regenerated plants may be self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present disclosure containing a desired polypeptide is cultivated using methods well known to one skilled in the art.

### EXAMPLES

The present disclosure is further illustrated in the following Examples, in which parts and percentages are by weight and degrees are Celsius, unless otherwise stated. It should be understood that these Examples, while indicating embodiments of the disclosure, are given by way of illustration only. From the above discussion and these Examples, one skilled in the art can ascertain the essential characteristics of this disclosure, and without departing from the spirit and scope thereof, can make various changes and modifications of the disclosure to adapt it to various usages and conditions. Thus, various modifications of the disclosure in addition to those shown and described herein will be apparent to those skilled in the art from the foregoing description. Such modifications are also intended to fall within the scope of the appended claims.

### Example 1

### Increase in Field Yield and Agronomic Characteristics of Maize Plants Expressing a DREB2 Polypeptide

Transgenic maize plants expressing a polynucleotide encoding TaDreb2 (SEQ ID NO: 1) were tested under various drought conditions at the field level. Constructs that included maize GOS2 promoter driving TaDreb2 gene.

TABLE 2
Yield increase of maize plants expressing SEQ ID NO: 1.

| | | | Percent |
|---|---|---|---|
| | | | Yield Change |
| | | | (Construct |
| Construct | | Stress | vs BN) |
| ZM-GOS2 PRO:: | Multi-Location; Multi- | SS | 0.79 |
| TADREB2 (MOD1) | tester; Multi-event | | |
| ZM-GOS2 PRO:: | Multi-Location; Multi- | MS | 11.76 |
| TADREB2 (MOD1) | tester; Multi-event | | |
| ZM-GOS2 PRO:: | Multi-Location; Multi- | OPT | 0.85 |
| TADREB2 (MOD1) | tester; Multi-event | | |

| | |
|---|---|
| All Locs | 6.10 |

Eight transgenic events were field tested at 4 primary geographical locations, with 11 total locations considering local replica 5 locations. Drought stress classifications included severe stress (SS), moderate stress (MS) and well-watered or optimal conditions (OPT). Controls included bulk null (BN)—seeds. Bulk nulls seeds involve a uniform randomized mixture of comparable hybrid seed for the event nulls (non-transgenic segregants) for each transgenic event in the trial. At one of the managed stress locations, mild drought conditions were imposed during flowering and severe drought conditions were imposed during the grain fill period (“grain fill stress). One of the locations was well-watered and another location experienced severe stress.

Yield data were collected in all locations with 3-6 replicates per location. Yield analysis was by ASREML (VSN International Ltd), and the values are BLUPs (Best Linear Unbiased Prediction) (Cullis, B. R et al. (1998) Biometrics 54: 1-18; Gilmour, A. R. et al. (2009) ASRemI User Guide 3.0; Gilmour, A. R., et al. (1995) Biometrics 51: 1440-50). Percent (%) increase over bulk null are shown in Table 2.

To analyze the yield data, a mixed model framework was used to perform the single and multi-location analysis.

In the single location analysis, main effect of construct is considered as a random effect. (However, construct effect might be considered as fixed in other circumstances). The main effect of event is considered as random. The blocking factors such as replicates and incblock (incomplete block design) within replicates are considered as random. Tester 1 and tester 2 were used where applicable.

Single_loc analysis across_loc analysis was performed and calculated blup (Best Linear Unbiased Prediction) for each event. The significance test between the event and BN was performed using a p-value of 0.1 in a two-tailed test. The significant values (with p-value less than or equal to 0.1 with a 2-tailed test) were calculated.

As shown in Table 2, the effect of the transgene on yield was mildly positive for the SS and OPT conditions on a construct level and substantially positive for the MS condition across all locations.

Effect of the transgene on other agronomic characteristics were also evaluated; such as plant height, reproductive parameters such as ASI (anthesis to silking interval), TTSLK (time to silking) and yield moisture (YLDMST) as shown in Table 3. Table 2 represents averages of multi-tester and multi-locations for the stress conditions indicated.

TABLE 3
Agronomic Characteristics of Maize
Plants Expressing SEQ ID NO: 1.

| | Percent | | | |
|---|---|---|---|---|
| | Plant | Percent | Percent | Percent |
| | Height | ASI | TTSLK | YLDMST |
| | Difference | Difference | Difference | Difference |
| | (Construct | (Construct | (Construct | (Construct |
| Stress | vs BN) | vs BN) | vs BN) | vs BN) |
| SS | −2.81 | NA | 0.75 | 8.44 |
| MS | −0.7 | −23.37 | −1.3 | 13.07 |
| OPT | −3.13 | −18.71 | −0.44 | 7.37 |
| All Locs | −2.75 | −22.4 | −0.71 | 8.06 |

The Table 3 describes transgenic testing results for TADREB2 for four agronomic metrics. The plant height was generally reduced for TADREB2 tested under severe, moderate or non-stressed locations, averaging about 2.75% decrease. Maintaining or increasing plant yield with the same or slightly shorter plants is considered a positive maize trait. The effect was fairly penetrant and independent of location. The ASI (anthesis to silking interval) is a metric of drought tolerance in maize. One component of ASI is the Time to Silking (TTSLK). Generally under drought stress the pollen is released much earlier than the silk, reducing fertilization, seed set and yield. A desirable drought trait is to reduce ASI by reducing the Time to Silking, allowing the male and female flowering times to overlap or ‘nick’. For TADREB2 lines, the ASI was substantially reduced, and this was associated with a reduction in Time to Silk. ASI and TTSK reduction was most prominent for moderate drought stress locations, where also the greatest yield benefit of TADREB2 was observed. The trait YLDMST (yield to moisture), is a metric dividing the yield by the grain moisture. Excess grain moisture can increase grain drying costs, reducing effective yield and profitability. For the TADREB2 lines the YLDMST trait remained positive, indicating the yield gain was not associated with excessive grain moisture.

### Example 2

### Transformation of Maize Using Agrobacterium

Maize plants can be transformed with the DNA construct containing ZmDREB2 or a DNA construct containing any of the corresponding homologs from maize (from Table 1) in order to examine the resulting phenotype.

Agrobacterium-mediated transformation of maize is performed essentially as described by Zhao et al. in Meth. Mol. Biol. 318:315-323 (2006) (see also Zhao et al., Mol. Breed. 8:323-333 (2001) and U.S. Pat. No. 5,981,840 issued Nov. 9, 1999, incorporated herein by reference). The transformation process involves bacterium inoculation, co-cultivation, resting, selection and plant regeneration.

### 1. Immature Embryo Preparation

Immature maize embryos are dissected from caryopses and placed in a 2 mL microtube containing 2 mL PHI-A medium.

### 2. Agrobacterium Infection and Co-Cultivation of Immature Embryos

### 2.1 Infection Step

PHI-A medium of (1) is removed with 1 mL micropipettor, and 1 mL of Agrobacterium suspension is added. The tube is gently inverted to mix. The mixture is incubated for 5 min at room temperature.

### 2.2 Co-Culture Step

The Agrobacterium suspension is removed from the infection step with a 1 mL micropipettor. Using a sterile spatula the embryos are scraped from the tube and transferred to a plate of PHI-B medium in a 100×15 mm Petri dish. The embryos are oriented with the embryonic axis down on the surface of the medium. Plates with the embryos are cultured at 20° C., in darkness, for three days. L-Cysteine can be used in the co-cultivation phase. With the standard binary vector, the co-cultivation medium supplied with 100-400 mg/L L-cysteine is critical for recovering stable transgenic events.

### 3. Selection of Putative Transgenic Events

To each plate of PHI-D medium in a 100×15 mm Petri dish, 10 embryos are transferred, maintaining orientation and the dishes are sealed with parafilm. The plates are incubated in darkness at 28° C. Actively growing putative events, as pale yellow embryonic tissue, are expected to be visible in six to to eight weeks. Embryos that produce no events may be brown and necrotic, and little friable tissue growth is evident. Putative transgenic embryonic tissue is subcultured to fresh PHI-D plates at two-three week intervals, depending on growth rate. The events are recorded.

### 4. Regeneration of T0 Plants

Embryonic tissue propagated on PHI-D medium is subcultured to PHI-E medium (somatic embryo maturation medium), in 100×25 mm Petri dishes and incubated at 28° C., in darkness, until somatic embryos mature, for about ten to eighteen days. Individual, matured somatic embryos with well-defined scutellum and coleoptile are transferred to PHI-F embryo germination medium and incubated at 28° C. in the light (about 80 μE from cool white or equivalent fluorescent lamps). In seven to ten days, regenerated plants, about 10 cm tall, are potted in horticultural mix and hardened-off using standard horticultural methods.

Media for Plant Transformation:
1. PHI-A: 4 g/L CHU basal salts, 1.0 mL/L 1000× Eriksson's vitamin mix, 0.5 mg/L thiamin HCl, 1.5 mg/L 2,4-D, 0.69 g/L L-proline, 68.5 g/L sucrose, 36 g/L glucose, pH 5.2. Add 100 μM acetosyringone (filter-sterilized).
2. PHI-B: PHI-A without glucose, increase 2,4-D to 2 mg/L, reduce sucrose to 30 g/L and supplemented with 0.85 mg/L silver nitrate (filter-sterilized), 3.0 g/L GELRITE®, 100 μM acetosyringone (filter-sterilized), pH 5.8.
3. PHI-C: PHI-B without GELRITE® and acetosyringonee, reduce 2,4-D to 1.5 mg/L and supplemented with 8.0 g/L agar, 0.5 g/L 2-[N-morpholino]ethane-sulfonic acid (MES) buffer, 100 mg/L carbenicillin (filter-sterilized).
4. PHI-D: PHI-C supplemented with 3 mg/L bialaphos (filter-sterilized).
5. PHI-E: 4.3 g/L of Murashige and Skoog (MS) salts, (Gibco, BRL 11117-074), 0.5 mg/L nicotinic acid, 0.1 mg/L thiamine HCl, 0.5 mg/L pyridoxine HCl, 2.0 mg/L glycine, 0.1 g/L myo-inositol, 0.5 mg/L zeatin (Sigma, Cat. No. Z-0164), 1 mg/L indole acetic acid (IAA), 26.4 μg/L abscisic acid (ABA), 60 g/L sucrose, 3 mg/L bialaphos (filter-sterilized), 100 mg/L carbenicillin (filter-sterilized), 8 g/L agar, pH 5.6.
6. PHI-F: PHI-E without zeatin, IAA, ABA; reduce sucrose to 40 g/L; replacing agar with 1.5 g/L Gelrite®; pH 5.6.

Plants can be regenerated from the transgenic callus by first transferring clusters of tissue to N6 medium supplemented with 0.2 mg per liter of 2,4-D. After two weeks the tissue can be transferred to regeneration medium (Fromm et al., Bio/Technology 8:833-839 (1990)).

Transgenic T0 plants can be regenerated and their phenotype determined. T1 seed can be collected.

Furthermore, a DNA construct can be introduced into an elite maize inbred line either by direct transformation or introgression from a separately transformed line.

Transgenic plants, either inbred or hybrid, can undergo more vigorous field-based experiments to study yield enhancement and/or stability under water limiting and water non-limiting conditions.

Subsequent yield analysis can be done to determine whether plants that contain the increased expression levels or increased activity of DREB2 genes have an improvement in yield performance (under stress or non-stress conditions), when compared to the control (or reference) plants that do not contain the DNA construct. Specifically, water limiting conditions can be imposed during the flowering and/or grain fill period for plants that have increased expression or activity levels of the DREB2 gene, and the control plants.

### Example 3

### Sequence Alignment and Percent Identity Calculations for DREB2 Polypeptides

Sequence alignments and percent identity calculations may be performed using the MEGALIGN® program of the LASERGENE® bioinformatics computing suite (DNASTAR Inc., Madison, Wis.). Multiple alignment of the sequences may be performed using the Clustal V method of alignment (Higgins and Sharp (1989) CABIOS. 5:151-153) with the default parameters (GAP PENALTY=10, GAP LENGTH PENALTY=10). Default parameters for pairwise alignments using the Clustal method are KTUPLE=1, GAP PENALTY=3, WINDOW=5 and DIAGONALS SAVED=5.

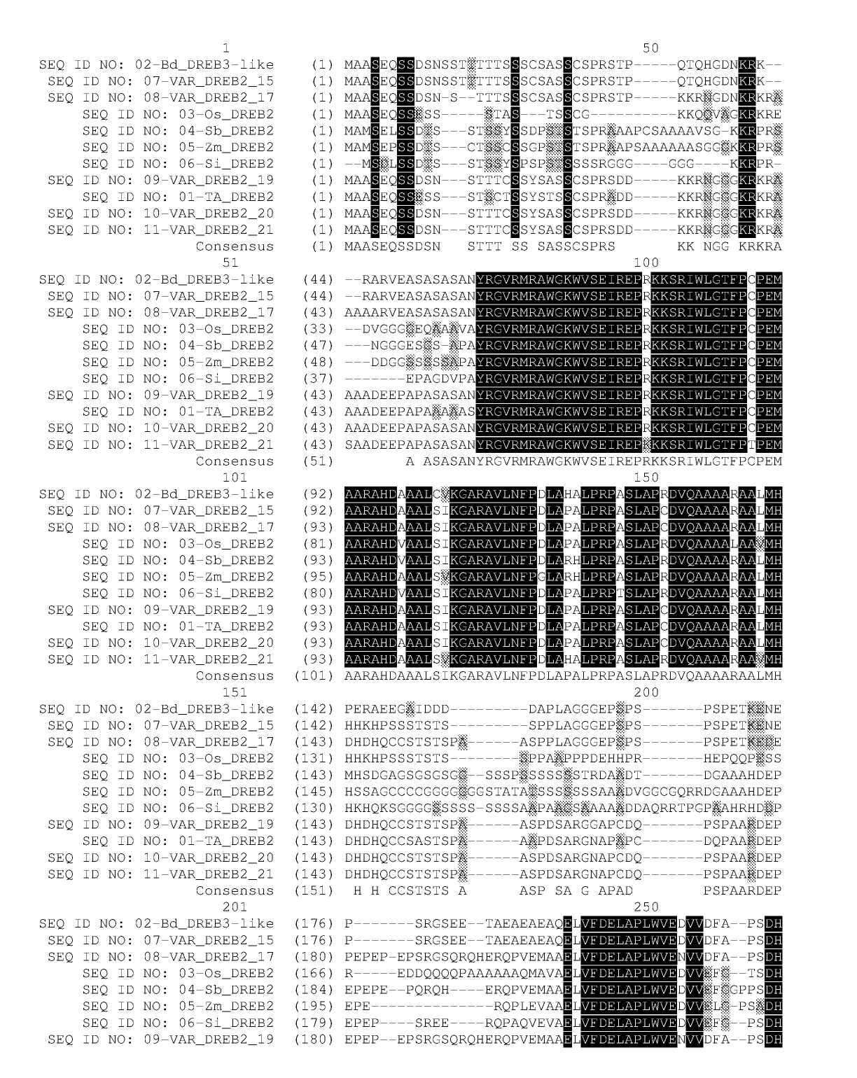

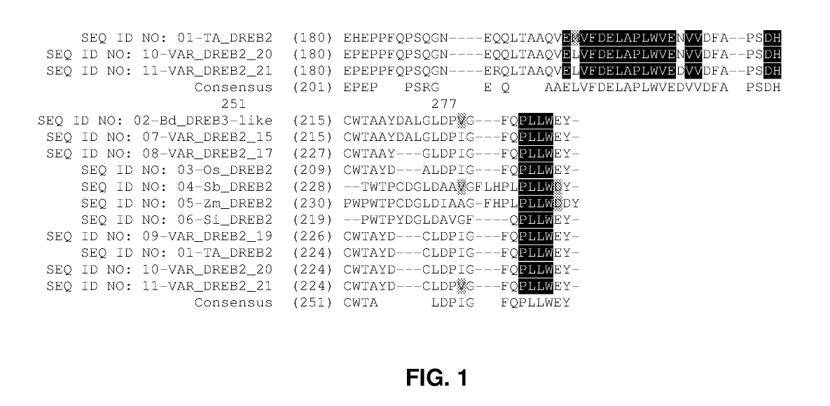

FIG. 1 shows the alignment of DREB2 polypeptides disclosed herein. This includes TaDREB2 (SEQ ID NO: 1) and its homologs and variants. Residues that are identical to the residue of consensus sequence at a given position are shown in a black background. A consensus sequence (black background) is presented where a residue is shown if identical in all sequences.

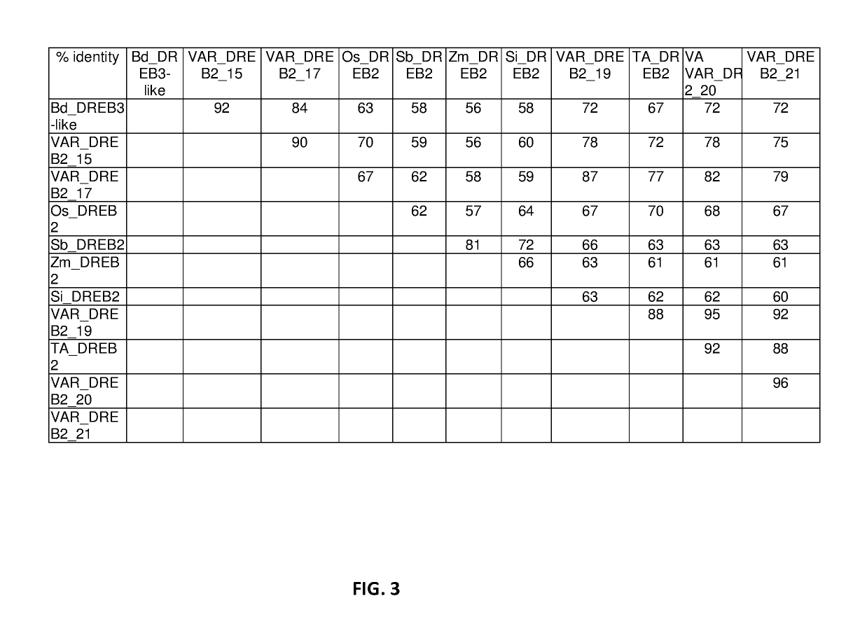

FIG. 3 shows the percent sequence identity and FIG. 2 the divergence values for each pair of amino acids sequences of DREB2 polypeptides displayed in FIG. 1.

What is claimed is:

1. A maize plant in which expression of a DREB2 gene is increased, when compared to a control plant, wherein the DREB2 gene encodes a DREB2 polypeptide and wherein the plant exhibits at least one phenotype selected from the group consisting of: increased yield, increased drought stress tolerance, and increased biomass compared to the control plant not expressing the DREB2 gene.

2. The maize plant of claim 1, wherein the DREB2 gene is TaDreb2.

3. The plant of claim 1, wherein the plant exhibits increased abiotic stress tolerance.

4. The plant of claim 1, wherein the DREB2 polypeptide comprises an amino acid sequence with at least 90% sequence identity to SEQ ID NOS: 1-11, 15, 17, 19, 21, 23, 25, and 27.

5. The plant of claim 1, wherein the plant exhibits the phenotype of increased yield and the phenotype is exhibited under non-stress conditions.

6. The plant of claim 1, wherein the plant exhibits the phenotype of increased yield and the phenotype is exhibited under stress conditions.

7. The plant of claim 1, wherein the plant exhibits the phenotype under drought stress conditions.

8. The plant of claim 4, wherein the increase in expression of an endogenous DREB2 gene encoding a polypeptide sequence comprising an amino acid sequence that is at least 95% identical to SEQ ID NO: 5 is caused by insertion of a heterologous regulatory element that increases the expression of the endogenous gene.

9. The plant of claim 4, wherein the increase in expression of an endogenous DREB2 gene encoding a polypeptide sequence comprising an amino acid sequence that is at least 95% identical to SEQ ID NO: 5 is caused by a mutation in the endogenous regulatory element that increases the expression of the endogenous DREB2 gene.

10. The plant of claim 8, wherein the endogenous DREB2 gene is altered by a zinc finger nuclease, Transcription Activator-Like Effector Nuclease (TALEN), CRISPR or meganuclease.

11. The plant of claim 1, wherein the activity of an endogenous DREB2 polypeptide is increased as a result of mutation of the endogenous DREB2 gene.

12. The plant of claim 11, wherein the mutation of the endogenous DREB2 gene is caused by zinc finger nuclease, Transcription Activator-Like Effector Nuclease (TALEN), CRISPR or meganuclease.

13. A DNA construct comprising a polynucleotide, wherein the polynucleotide encodes a polypeptide comprising a sequence that is at least 95% identical to SEQ ID NO: 1, is operably linked in sense orientation to a heterologous promoter, wherein the expression of the polynucleotide in a maize results in an increased yield of at least about 5% as compared to a control plant not expressing the polypeptide.

14. The DNA construct of claim 13, wherein the heterologous promoter is maize GOS2 promoter.

15. The DNA construct of claim 14, wherein the heterologous promoter hybridizes under stringent conditions to a sequence that comprises SEQ ID NO: 14.

16. A method of making a plant in which expression of an endogenous DREB2 gene is increased, when compared to a control plant, and wherein the plant exhibits at least one phenotype selected from the group consisting of: increased yield, increased abiotic stress tolerance, and increased biomass, compared to the control plant, the method comprising the steps of introducing into a plant a DNA construct comprising a polynucleotide operably linked to a heterologous promoter, wherein the DNA construct is effective for increasing expression of an endogenous DREB2 gene.

17. The method of claim 16, wherein the DNA construct comprises a heterologous regulatory element.

18. The method of claim 17, wherein the heterologous regulatory element is a promoter.

19. The method of claim 17, wherein the heterologous regulatory element is maize GOS2 promoter.

20-23. (canceled)

24. A plant comprising the DNA construct of any of the claims 13-15, wherein expression of the DREB2 gene is increased in the plant, when compared to a control plant, and wherein the plant exhibits at least one phenotype selected from the group consisting of: increased yield, increased abiotic stress tolerance and increased biomass, compared to the control plant.

25. The plant of claim 24, wherein the plant exhibits an increase in abiotic stress tolerance, and the abiotic stress is drought stress, low nitrogen stress, or both.

26. The plant of claim 24, wherein the plant is a monocot plant.

27. The plant of claim 26, wherein the monocot plant is a maize plant.

28-31. (canceled)
